(19) United States
(12) Patent Application Publication
(10) Pub. No.: US 2007/0015132 A1
(43) Pub. Date: Jan. 18, 2007

(54) System and methods for measuring at least one metabolic rate of a plurality of cells

(75) Inventors: Robert Balcarcel, Nashville, TN (US); Lindsey Clark, Nashville, TN (US); Yuansheng Yang, Nashville, TN (US); Franz J. Baudenbacher, Franklin, TN (US); Owen McGuineness, Brentwood, TN (US); Ales Prokop, Nashville, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: VANDERBILT UNIVERSITY

(21) Appl. No.: 10/483,540

(22) PCT Filed: Aug. 6, 2002
(86) PCT No.: PCT/US02/25113

§ 371 Date: Sep. 18, 2006

Related U.S. Application Data
(60) Provisional application No. 60/310,652, filed on Aug. 6, 2001.

(51) Int. Cl. C12Q 1/00; C12Q 1/30

(52) U.S. Cl. 435/4; 435/27

(57) ABSTRACT

A system and methods for calculating at least one unknown metabolic flux of a plurality of cells. In one embodiment, the method includes the steps of constructing a metabolic network having a plurality of reaction components, the reaction components representing at least glycolysis, reduction of pyruvate to lactate, TCA cycle, and oxidative phosphorylation, measuring at least two metabolic rates of a plurality of cells corresponding to at least two of the metabolic network reactions, and calculating metabolic fluxes of a plurality of cells for the rest of the metabolic network reactions from at least two measured metabolic rates of a plurality of cells corresponding to at least two of the reactions.

The present invention was made with Government support under Grant No. N66001-01-C-8064 awarded by the Defense Advanced Research Projects Administration. The United States Government may have certain rights to this invention pursuant to these grants.

This application is being filed as a PCT International Patent application in the name of Vanderbilt University, a U.S. national corporation, Applicant for all designated countries except the US, and Robert Balcercel, a U.S. citizen and resident, Applicant for the designation of the US only, on 6 Aug. 2002.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

### FIELD OF THE INVENTION

The present invention generally relates to a system and methods for measuring at least one metabolic rate of a plurality of cells. More particularly, the present invention relates to a system and methods that utilize a first well plate and a second well plate to measure the concentration of at least one metabolite of a plurality of cells and determine at least one metabolic rate therefrom.

Certain embodiments of the present invention comprise system and methods for calculating at least one unknown metabolic flux of a plurality of cells. In one embodiment, the method includes the steps of constructing a metabolic network having a plurality of reaction components, the reaction components representing at least glycolysis, reduction of pyruvate to lactate, TCA cycle, and oxidative phosphorylation, measuring at least two metabolic rates of a plurality of cells corresponding to at least two of the metabolic network reactions, and calculating metabolic fluxes of a plurality of cells for the rest of the metabolic network reactions from at least two measured metabolic rates of a plurality of cells corresponding to at least two of the reactions.

### BACKGROUND OF THE INVENTION

The biological cell may act as a parallel processing, non-linear, multistate, analog computer. This analog computer can occupy a volume of less than 10⁻¹⁶ m³ and is primarily powered only by sugars, fats, and oxygen. The complexity of these computers is evidenced by the attempts to model ongoing biochemical processes based on Mycoplasma genitalium, a microbe with the smallest known gene set of any self-replicating organism (http:\\www.e-cell.org). However, even this simplest model requires hundreds of variables and reaction rules, and a complete model even for a mammalian cell would be much more complex, requiring in excess of 10⁵ variables and equations.

In recent years, with the threats posed by toxicants or military concern growing, the conventional detection technologies, most of which rely on structural recognition or other aspects of chemical structure, can not satisfy the daunting task of detecting and interpreting the significance of these often chemically diverse threats, the demand for developing a new kind of technology which address wide spectrum activity detection, rather than molecular recognition, is becoming increasingly necessary.

In the past decades, numerous biosensors have been developed and implemented for toxic substance detection. High specificity and sensitivity are obtained by using binding components (enzymes, antibodies, nucleic acids, DNA, receptors) as biological sensing. However, the inherent instability of proteins, the lack of suitable binding components, and the requirement of knowledge of the structure and chemistry of the detected materials significantly limit the utilization of this kind of biosensor for wide-spectrum detection. Of the multitude of toxic materials concerned, only a small number can be detected by the currently developed biosensors.

In recent years, a new kind of class of biosensors has emerged based on the ability to interrogate cellular or tissue microarrays. Ability of yielding insight into functional information can be obtained by monitoring physiologic, metabolic, or network processes and response of cells and tissues. In contrast to the binding components biosensor, physiological impacts of toxicants are sought instead of the identity of the toxicants themselves. Information at cellular level enables not only detection but also classification, and offers the potential of rapid and wide-spectrum detection of known or even unknown toxicants; further investigation on metabolism will provide some information about toxicant action mechanism.

One major challenge of biological activity biosensor is to develop sound methods for achieving clear signatures of the impact of toxicants. Using unique characteristics specific to some cells, such as membrane potential, bioluminescence, morphology, and photosynthetic activity, many whole cell-based biosensors have been developed for toxicant detection. The major drawbacks of these kinds of biosensors are the difficulty in interpreting the signals and the utilization of specific cells.

Another alternative to monitor metabolic state of cells is to measure metabolite metabolic rates, which can provide not only direct evidence of toxicant action but also some information related to toxicant mechanism. There are very few reports about toxicant detection through monitoring metabolic rate. Based on the recognition that Hep G2 has many receptors on its membrane for uptaking LDL (low density lipoprotein), a bioassay method using LDL uptake rate as a novel index of metabolic activity has been developed for monitoring the cytotoxicity of environmental pollutants.

Cellular processes are metabolically driven, energy requiring events. The overall result of the totality of cellular reaction is the conversion of nutrients into free energy and metabolic products. Both lactate produced by glycolysis at anaerobic conditions and CO₂ produced through respiration at aerobic condition lower media pH. Thus, media acidification rate is coupled tightly to the rates of cell metabolism. Introduction of Cytosensor microphysiometer enables rapid and precise measurement of extracellular acidification rate in real time. Evaluation of in vitro cytotoxicity of toxicants by measuring medium acidification rate with the Cytosensor microphysiometer has been reported.

However, most biosensors at present can only measure one parameter, and each time only one independent measurement can be done. These devices can not satisfy the high throughput requirement in toxicant detection and drug screening in pharmacology. Moreover, monitoring one parameter enables only evaluation of cytotoxicity. For toxicant discrimination, classification or even mechanism determination, measuring more parameters is needed.

Therefore, among other things, there is a need to develop new system and methods that are capable of measuring multiple metabolite or parameters during a single operation or experiment.

### SUMMARY OF THE INVENTION

In one aspect, the present invention relates to a method for measuring at least one metabolic rate of a plurality of cells. In one embodiment, the method includes the step of providing a first plate having a plurality of wells, wherein the total number of the plurality of wells is L, L being an integer. Each well has a bottom and side portions in cooperation defining a volume and an opening opposite the bottom. The method further includes the steps of placing a solution of medium and cells in one or more wells of the first plate, wherein the amount of solution in each well in terms of volume is v₀, and withdrawing a first volume, v₁, of medium with or without cells from one or more wells of the first plate, thereby leaving a second volume, v₂, of medium and cells in one or more wells of the first plate. The method additionally includes the steps of incubating the first plate for a period of time, T₁, withdrawing a third volume, v₃, of medium with or without cells, from one or more wells of the first plate, thereby leaving a fourth volume, v₄, of medium and cells in one or more wells of the first plate, withdrawing a fifth volume, v₅, of medium with cells, from one or more wells of the first plate, thereby leaving a sixth volume, v₆, of medium and cells in one or more wells of the first plate, obtaining cell-free solutions from the first and third volumes, using the cell-free solutions in an assay, measuring the concentration of at least one metabolite in the first and third volumes or in the second volume at least two times within a time period T₂, wherein T₂ is less than or equal to T₁ and within time period T₁, and determining at least one metabolic rate for the metabolite measured for each of one or more wells of the first plate that contained a plurality of cells from the measured concentration of at least one metabolite.

In one embodiment, the first plate includes a well-plate and L is 24. The original volume v₀ is smaller than 1,000 μl. As used herein, “cell” or “cells” represent any biologically active entity, including but not limited to ex vivo tissue samples, artificial tissues, bacterial cells, yeast cells, mammalian cells, in vitro enzyme systems, and cellular components such as mitochondria and ribosomes. Moreover, “medium” represents any liquid phase that supports the biological entity to be measured, including but not limited to serum-based medium, serum-free medium, protein-free medium, ringer's solution, basal salt solutions, and custom medium.

The cells can grow in suspension remaining unattached from the bottom or side surfaces. For this situation, the step of obtaining cell-free solutions includes the step of centrifugating the first volume and the third volume, respectively. Alternatively, the cells can grow attached to the bottom or side portions of the well or on a device placed in the well. Then, the step of obtaining cell-free solutions may include the step of avoiding the cells attached to the bottom or side portions of the well or a device placed in the well, wherein the device placed in the well can be a scaffold or at least one microcarrier.

Additionally, prior to the step of withdrawing a first volume, further including the step of keeping the solution and the cells in one of more wells of the first plate for a period of time, T₃, wherein T₃ is sufficiently long to allow adherent cells to attach to a surface of a corresponding well or a device placed therein.

The incubating step further includes the step of placing the first plate in an incubator, which provides proper temperature, humidity, and gas phase carbon dioxide control.

In one embodiment, prior to the step of placing a solution of medium and cells in one of more wells of the first plate, the method further includes the step of preparing the solution of medium and cells in a parent culture, where centrifuging and changing medium can be made as needed to achieve a desired test environment and a desired concentration of cells.

Additionally, subsequent to the step of obtaining cell-free solutions, the method further includes the step of storing the cell-free solutions for later use. The cell-free solutions can be stored in a refrigerator. Or, the cell-free solutions can be stored in a freezer.

Moreover, subsequent to the step of withdrawing the fifth volume, the method further includes the steps of performing a cell count to determine cell concentration and culture viability from a portion of the fifth volume, performing an assay for apoptosis and necrosis, or performing a cellular or molecular biology assay.

In one embodiment, the method allows a plurality of metabolic rates of the cells to be determined at the same time, where the total number of the plurality of metabolic rates is an integer Q greater than one. At least one of the plurality of metabolic rates is for consumption or production of glucose, lactate, any of amino acids, oxygen, carbon dioxide, hydrogen ion (pH), or biopharmaceutical.

In one embodiment, the solution of medium and cells in each well of the first plate has a cell density substantially similar to each other. The cell density of the solution of medium and cells in each well of the first plate is in the range of 1.0×10⁴ to 1.0×10⁹ cells/ml. For example, the cell density of the solution of medium and cells has a concentration of cells of about 2.0×10⁶ cells/ml. The method of claim 21, wherein the amount of biological entity in the solution is in the range of 0.0001 to 2000 grams/liter. Alternatively, the solution of medium and cells in each well of the first plate has a cell concentration different from each other. Note that a number of cells and/or an amount of medium can be supplied to each well of the first plate during operation.

The method further includes the step of analyzing the first and third volumes obtained from each well for at least one metabolite concentration, which can be accomplished by the following steps providing at least one second plate having a plurality of wells, each well having a bottom and side portions in cooperation defining a volume and an opening opposite the bottom, wherein the total number of the plurality of wells is M, M being an integer larger than L, and placing portions of the solution from one or more volumes obtained from the first plate into each of S wells of at least one second plate, wherein each of S wells of at least one second plate contains a reagent solution for accomplishing a particular metabolite assay for R times, where R is an integer and S is an integer smaller than M.

In doing so, the volumes of the solution used can be the first volumes from the first plate, and the number of wells needed in at least one second plate is no greater than R×L, where each volume is apportioned R times. Also, the volumes of the solution can be the third volumes from the first plate, and the number of wells needed in at least one second plate is no greater than R×L where each volume is apportioned R times. Moreover, the volumes of the solution can be both the first and third volumes from the first plate, and number of wells needed in at least one second plate is no greater than 2×R×L, where each volume is apportioned R times.

When doing so, the metabolite analyzed can be glucose and the reagent solution contains enzymes and substrates that use glucose to create NADPH. Moreover, the metabolite analyzed can be lactate and the reagent solution contains enzymes and substrates that use lactate to create NADH. And the metabolite analyzed can be carbon dioxide and bicarbonate and the reagent solution contains enzymes and substrates that use bicarbonate to oxidize NADH.

In one embodiment, M is at least three times larger than L. For example, one choice is that L is 24 and M is 96. Other choices of L and M can also be made to practice the present invention. Moreover, R is chosen as 3 for an example. R can be other numbers such as 1, 2, 4 or the like.

Furthermore, prior to the step of withdrawing a first volume, the method further includes the step of monitoring the pH of each well in the first plate by spectroscopy for a time period T₄, which is less than or equal to T₂, wherein one or more wells of the first plate are sealed during T₄. Additionally, prior to the step of withdrawing a first volume, the method further includes the step of monitoring the oxygen concentration of each well by spectroscopy for a time period T₅, which is less than or equal to T₂, and may overlap with or coincide with T₄, wherein one or more wells of the first plate are sealed during T₅.

Optionally, the method further includes the step of sampling a seventh volume, v₇, and an eighth volume, v₈, from one or more wells of the first plate immediately before and immediately after a period of time, T₆, which is less than or equal to T₂ in length, and may overlap with or coincide with at least one of T₄ and T₅, to leave volumes v₉ and v₁₀ in one or more wells of the first plate, respectively, wherein one or more wells of the first plate are sealed during a period of time T₆.

Moreover, the determining step further includes the step of determining at least one or more amino acids from portions of the first and third cell-free volumes, wherein the step of determining at least one or more amino acids further includes the step of determining amino acids by using a liquid chromatography system such as an LC or HPLC system. The determining step also includes the step of determining biopharmaceutical concentration from portions of the first and third cell-free volumes, wherein the biopharmaceutical includes at least one of a monoclonal antibody and a therapeutic protein.

In another aspect, the present invention relates to method for calculating at least one unknown metabolic flux of a plurality of cells. In one embodiment, the method includes the steps of constructing a metabolic network having a plurality of reaction components, the reaction components representing at least glycolysis, reduction of pyruvate to lactate, TCA cycle, and oxidative phosphorylation, measuring at least two metabolic rates of a plurality of cells corresponding to at least two of the metabolic network reactions, and calculating metabolic fluxes of a plurality of cells for the rest of the metabolic network reactions from at least two measured metabolic rates of a plurality of cells corresponding to at least two of the reactions.

Moreover, the method includes the steps of measuring at least one additional metabolic rates of a plurality of cells corresponding to an additional one of the reactions, constructing a set of equations that are overdetermined for the metabolic rates of a plurality of cells for the reaction components, and calculating metabolic fluxes of a plurality of cells for all of the reactions from the set of equations.

Additionally, the method further includes the step of feedbacking the measured at least two metabolic rates of a plurality of cells corresponding to two of the reaction components from the determined metabolic rates, wherein the plurality of reaction network components include glucose, pyruvate, lactate, CO₂, O₂, ATP, NADH, FADH₂, and amino acids, and wherein measurable reaction fluxes include glucose, lactate, oxygen, and carbon dioxide metabolic rates, and calculated fluxes include glycolysis, TCA cycle, oxidative phosphorylation, and ATP production.

In yet another aspect, the present invention relates to a system for calculating at least one unknown metabolic flux of a plurality of cells. In one embodiment, the system includes means for constructing a metabolic network having a plurality of reaction components, the reaction components representing at least glycolysis, reduction of pyruvate to lactate, TCA cycle, and oxidative phosphorylation, means for measuring at least two metabolic rates of a plurality of cells corresponding to at least two of the metabolic network reactions, and means for calculating metabolic fluxes of a plurality of cells for the rest of the metabolic network reactions from at least two measured metabolic rates of a plurality of cells corresponding to at least two of the reactions.

Moreover, the system includes means for measuring at least one additional metabolic rates of a plurality of cells corresponding to an additional one of the reactions, means for constructing a set of equations that are overdetermined for the metabolic rates of a plurality of cells for the reaction components, and means for calculating metabolic fluxes of a plurality of cells for all of the reactions from the set of equations.

Additionally, the system further includes means for feedbacking the measured at least two metabolic rates of a plurality of cells corresponding to two of the reaction components from the determined metabolic rates, wherein the plurality of reaction network components include glucose, pyruvate, lactate, CO₂, O₂, ATP, NADH, FADH₂, and amino acids, and wherein measurable reaction fluxes include glucose, lactate, oxygen, and carbon dioxide metabolic rates, and calculated fluxes include glycolysis, TCA cycle, oxidative phosphorylation, and ATP production.

In one embodiment, the measuring means includes a first well plate having a plurality of wells, each well having a bottom and side portions in cooperation defining a volume and an opening opposite the bottom, wherein the total number of the plurality of wells is L, L being an integer. Moreover, the measuring means further includes a second well plate having a plurality of wells, each well having a bottom and side portions in cooperation defining a volume and an opening opposite the bottom, wherein the total number of the plurality of wells is M, M being an integer that is same as or different from L. Additionally, the calculating means includes a controller that can be associated with a computer. Moreover, one or more computer can be utilized to automate the system and processes according to the present invention, which makes measuring multiple metabolite or parameters during a single operation or experiment into a reality. Note that various types of sensors can be placed into the wells to monitor the status of the cells and make dynamic measurements, which allows the present invention to be practiced in a lot of areas.

These and other aspects will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 schematically shows a well plate used for the metabolic screening method according to one embodiment of the present invention: A. top view; and B. side view.

FIG. 2 schematically show a top view of a microtiter plate used for the metabolite assays according to one embodiment of the present invention.

FIG. 3 schematically shows a flow chart of the well plate process for metabolic screening of cells according to one embodiment of the present invention.

FIG. 3A schematically shows an overview and timeline for the metabolic screening method according to one embodiment of the present invention as shown in FIG. 3.

FIG. 4 shows a Simplified Metabolic Network according to one embodiment of the present invention.

FIG. 5 shows a Detailed Metabolic Network according to one embodiment of the present invention.

FIG. 6 shows comparison of changes in energy production. (A) ATP production flux calculated from either Model 1 (▪) or Model 2 (Δ), and (B) percent of ATP produced by glycolysis and TCA cycle. Letters refer to steady state cultures obtained in a bioreactor system described previously. Percent ATP from glycolysis calculated from fluxes v₁, v₂, and v₆ for Model 1 (□), and v₁ for Model 2 (Δ). Percent ATP from TCA cycle was calculated from fluxes v₂₀, v₂₁, v₂₄, v₂₅, v₂₇, and v₈₀ for Model 1 (▪), and v₂₀ for Model 2 (▴). NAD(P)H and FADH₂ associated with these pathways was converted to ATP using stoichiometries of 2.5 and 1.5 respectively.

FIG. 7 shows comparison of changes in flux distribution. (A) Percent flux through glycolysis and TCA cycle on a 6-carbon basis. For Model 1, glycolysis (▪) is the average of fluxes v₁, v₂, and v₆/2 and TCA cycle (□) is the sum of the CO₂ evolved from fluxes v₂₀, v₂₁, v₂₄, and v₈₀, divided by 6. For Model 2, glycolysis (Δ) is v₁ and TCA cycle (▴) is v₂₀/2. (B) Lactate/Glucose ratios on a 6-carbon basis. Values for rates used to compute L/G ratios from models 1 (▪) and 2 (Δ) were fluxes v₁ and v₁₁/2 in both cases. The measured rates for q_{glc} and q_{lac}/2 were used to calculate the observed L/G ratio (□).

FIGS. 8A-B illustrate measured metabolic rates from 24-well plates obtained during screening of rapamycin. (A) Glucose and (B) lactate rates for each well of a 24-well plate HTMS experiment. Error bars are those propagated from noise associated with concentration difference and cell density measurements.

FIGS. 9A-B show average measured metabolic rates during rapamycin screening. (A) Glucose uptake (▪) and lactate production (□) rates. (B) Lactate-to-glucose ratio on a 6-carbon basis. Error bars are the standard deviation of rates from 4 wells in a concentration group. Rates from every well were included. Numbers shown are p-values from a 2-tailed t-test comparing a particular rapamycin concentration with the control.

FIGS. 10A-B show comparison of changes in energy production. q_{ATP} (A) and percent of ATP from TCA cycle (B), each versus concentration of rapamycin. q_{ATP} values are the average of values estimated for each individual well using Model 2, with glucose and lactate rates as inputs. Percent ATP is estimated for each well, as described in FIG. 3. All error bars shown are the standard deviation among wells of same concentration group. Numbers above each data bar or point are p-values for 2-tailed t-test vs. the control. T-test could not be performed for 250 nM as data from 3 of 4 wells was excluded.

FIGS. 11A-B schematically show comparison of changes in flux distribution. (A) Percent carbon flux through TCA cycle and (B) L/G ratio, each versus concentration of rapamycin. Flux through TCA and L/G ratio for each well is determined as in FIG. 4. Numbers above each data point are p-values for 2-tailed t-test vs. the control. T-test could not be performed for 250 nM as data of 3 of 4 wells was excluded.

FIG. 12 schematically shows proportionally diluted fibroblast cells attached in 24-well plate read by U/V spectrophotometer and plotted vs. cell density. At every point mean values and standard deviations for 4 measurements are shown.

FIG. 13 shows pH monitoring of test medium during fibroblast cells exposed to 2,4-dinitrophenol at different concentrations. Measured pH values are shown as mean values of four measurements with their standard deviation. Blank pH dropping because of CO₂ dissolved in the medium.

FIG. 14 illustrates effects of four kinds of toxicants on the medium acidification rate of fibroblast cell. At every point, mean values and standard deviation for four measurements are shown.

FIG. 15 illustrates impact of fluoride on the glucose (▪), lactate (▴) metabolic rate, and medium acidification rate (♦). At every point, mean values and standard deviation of four.

FIG. 16 illustrates impact of 2,4-dinitrophenol (DNP) on the glucose (▪), lactate (▴) metabolic rate, and medium acidification rate (♦). At every point, mean values and standard deviation of four measurements are shown.

### DETAILED DESCRIPTION OF THE INVENTION

Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Additionally, some terms used in this specification are more specifically defined below.

### Definitions

The terms used in this specification generally have their ordinary meanings in the art, within the context of the invention, and in the specific context where each term is used. For example, conventional techniques of molecular biology, microbiology and recombinant DNA techniques may be employed in accordance with the present invention. Such techniques and the meanings of terms associated therewith are explained fully in the literature. See, for example, Sambrook, Fitsch & Maniatis. Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994). See also, PCR Protocols: A Guide to Methods and Applications, Innis et al., eds., Academic Press, Inc., New York (1990); Saiki et al., Science 1988, 239:487; and PCR Technology: Principles and Applications for DNA Amplification, H. Erlich, Ed., Stockton Press.

Certain terms that are used to describe the invention are discussed below, or elsewhere in the specification, to provide additional guidance to the practitioner in describing the devices and methods of the invention and how to make and use them. For convenience, certain terms are highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

The term “agent” is broadly defined as anything that may have an impact on any living system such as a cell. For examples, the agent can be a chemical agent. The chemical agent may comprise a toxin. The agent can also be a biological agent. Moreover, the agent may comprise at least one unknown component, which may be identified by practicing the present invention. Additionally, the agent may comprise at least one known component, whose interaction with cells or other components of an environment may be detected by practicing the present invention. The agent can also be a physical agent. Other examples of agent include biological warfare agents, chemical warfare agents, bacterial agents, viral agents, other pathogenic microorganisms, emerging or engineered threat agents, acutely toxic industrial chemicals (“TICS”), toxic industrial materials (“TIMS”) and the like. Examples of chemical agents that may be related to practicing the present invention include Mustard (that may be simulated with chloroethyl ethyl sulphide (endothelia cells in PC)), GB-Sarin (that may be simulated with Disopropylfluorophosphate (DFP)), VX (that may be simulated with Malathion) or the like. Examples of viral agents (and their simulants) that may be related to practicing the present invention include MS2, Hepatitus or simulant or attenuated virus, Retroviruses alphaviruses find set or the like. Examples of bacterial agents (and their simulants) that may be related to practicing the present invention include Bacillus globigii or Bacillus subtilis as spore formers similar to anthrax, Erwinia herbicola as a simulant for vegetative bacteria (not sporagenic), E. coli or the like.

The term “toxin” is broadly defined as any agent that may have a harmful effect or harmful effects on any living system such as a cell. Examples of toxins that may be related to practicing the present invention include cyanide, endotoxin, okadaic acid, Phorbol Myristate Acetate (“PMA”), microcystin, Dinitrophenol (“DNP”), Botulinum toxin (a common threat agent; inhibit transmitter release, whole cell MB), Staphylococcus enterotoxin B, ricin (inhibits protein synthesis and ribosmone, OT), mycotoxins, aflatoxins, cholera toxin (activates Cl pump, vesicle MB, NBR), Saxatoxin or tetrodotoxin (Na channel blocker, vesicle MB), Microcystins (hepatocyte metabolism in PC) and organophosphates. Other examples of toxins may be also discussed somewhere else in the specification. Additional examples of toxins can also be found in the market.

The term “molecule” means any distinct or distinguishable structural unit of matter comprising one or more atoms, and includes for example polypeptides and polynucleotides.

“DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases.

As used herein, “cell” means any cell or cells, as well as viruses or any other particles having a microscopic size, e.g. a size that is similar to that of a biological cell, and includes any prokaryotic or eukaryotic cell, e.g., bacteria, fungi, plant and animal cells. Cells are typically spherical, but can also be elongated, flattened, deformable and asymmetrical, i.e., non-spherical. The size or diameter of a cell typically ranges from about 0.1 to 120 microns, and typically is from about 1 to 50 microns. A cell may be living or dead. As used herein, a cell is generally living unless otherwise indicated. As used herein, a cell may be charged or uncharged. For example, charged beads may be used to facilitate flow or detection, or as a reporter. Biological cells, living or dead, may be charged for example by using a surfactant, such as SDS (sodium dodecyl sulfate). Cell or a plurality of cells can also comprise cell lines. Example of cell lines include liver cell, macrophage cell, neuroblastoma cell, endothelial cell, intestine cell, hybridoma, CHO, fibroblast cell lines, red blood cells, electrically excitable cells, e.g. Cardiac cell, myocytes (AT1 cells), cells grown in co-culture, NG108-15 cells (a widely used neuroblastoma X glioma hybrid cell line, ATCC# HB-12317), primary neurons, a primary cardiac myocyte isolated from either the ventricles or atria of an animal neonate, an AT-1 atrial tumor cardiac cell, Liver cells are also known as Hepatocytes, Secretory cell (depolarize and it secretes things) pancreatic beta cells secrete insulin, HELA cells (Helen Lane), HEK293 Human Epithial Kidney c, Erythrocytes (primary red blood cells), Lymphocytes and the like. Each cell line may include one or more cells, same or different. For examples, the liver cell comprises at least one of Human hepatocellular carcinoma (“HEPG2”) cell, CCL-13 cell, and H4IIE cell, the macrophage cells comprises at least one of peripheral blood mononuclear cells (“PBMC”), and skin fibroblast cells, the neuroblastoma cell comprises a U937 cell, the endothelial cell comprises a human umbilical vein-endothelial cell (“Huv-ec-c”), and the intestine cell comprises a CCL-6 cell.

A “reporter” is any molecule, or a portion thereof, that is detectable, or measurable, for example, by optical detection. In addition, the reporter associates with a molecule or cell or with a particular marker or characteristic of the molecule or cell, or is itself detectable, to permit identification of the molecule or cell, or the presence or absence of a characteristic of the molecule or cell. In the case of molecules such as polynucleotides such characteristics include size, molecular weight, the presence or absence of particular constituents or moieties (such as particular nucleotide sequences or restrictions sites). The term “label” can be used interchangeably with “reporter”. The reporter is typically a dye, fluorescent, ultraviolet, or chemiluminescent agent, chromophore, or radio-label, any of which may be detected with or without some kind of stimulatory event, e.g., fluoresce with or without a reagent. Typical reporters for molecular fingerprinting include without limitation fluorescently-labeled single nucleotides such as fluorescein-dNTP, rhodamine-dNTP, Cy3-dNTP, Cy5-dNTP, where dNTP represents DATP, dTTP, dUTP or dCTP. The reporter can also be chemically-modified single nucleotides, such as biotin-dNTP. Alternatively, chemicals can be used that react with an attached functional group such as biotin.

A “marker” is a characteristic of a molecule or cell that is detectable or is made detectable by a reporter, or which may be coexpressed with a reporter. For molecules, a marker can be particular constituents or moieties, such as restrictions sites or particular nucleic acid sequences in the case of polynucleotides. The marker may be directly or indirectly associated with the reporter or can itself be a reporter. Thus, a marker is generally a distinguishing feature of a molecule, and a reporter is generally an agent which directly or indirectly identifies or permits measurement of a marker. These terms may, however, be used interchangeably.

A “measurable quantity” is a physical quantity that is measurable by a device, or obtainable by simulations. For examples, a measurable quantity can comprise a physical quantity related to cellular physiological activities of a cell exposed to an agent. Because cellular physiological activities of a cell involve a lot of activities across a wide spectrum, the plurality of physical quantities related to the impact of the agent on the cell physiology of the cell exposed to the agent are numerous such as heat production, oxygen consumption, uncoupling ratio between heat production and oxygen consumption, free radical synthesis, fraction of oxygen diverted to free radical synthesis, reduced nicotinamide adenine dinucleotide phosphate (“NAD(P)H”), acid production, glucose uptake, lactate release, gluconeogenesis, transmembrane potential, intracellular messengers, membrane conductance, transmembrane pump and transporter rates, messenger RNA expression, neurotransmitter secretion, intracellular glycolytic stores, transmembrane action potential amplitude and firing rate, heat-shock protein expression, intracellular calcium, calcium spark rate and the like.

The term “channel” is broadly defined as any ionic pathway that is associated with cellular physiological activities of a cell. There are various types of channels. For examples, a channel can be a Voltage-gated channel, a Ligand-gated channel, Resting K+ channels (that are inwardly rectifying K, leak channels), Stretch activated channels, Volume-regulated channels and the like. Examples of Voltage-gated channel include K, Na, Ca and Cl. Examples of Ligand-gated channel include Neurotranmitter (glutamate {NMDA, AMPA, KAINATE}, GABA, ACH (nicotinic), 5HT, glycine, histamine, Cyclic nucleotide-gated (cAMP, cGMP from inside of cell), some K-selective, some non-specific cation channels, G-protein activated (mostly potassium; pertussis toxin-inhibited), Calcium-activated (K channels activated by voltage and Ca) and the like.

A “sensor” is broadly defined as any device that can measure a measurable quantity. For examples, a sensor can be a thermal detector, an electrical detector, a chemical detector, an optical detector, an ion detector, a biological detector, a radioisotope detector, an electrochemical detector, a radiation detector, an acoustic detector, a magnetic detector, a capacitive detector, a pressure detector, an ultrasonic detector, an infrared detector, a microwave motion detector, a radar detector, an electric eye, an image sensor, any combination of them and the like. A variety of sensors can be chosen to practice the present invention.

A “controller” is broadly defined as any device that can receive, process and present information. For examples, a controller can be one microprocessor, several microprocessors coupled together, a computer, several computers coupled together, and the like.

The term “biosignature” means a marker for a particular signaling or metabolic pathway affected by an agent.

The term “analyte” means a material that can be consumed or produced by a cell. Examples of analyte of interest include pH, K, oxygen, lactate, glucose, ascorbate, serotonin, dopamine, ammonina, glutamate, purine, calcium, sodium, potassium, NADH, protons, insulin, NO (nitric oxide) and the like.

A “medium” is a fluid that may contain one or more agents, one or more analytes, or any combination of them. A medium can be provided with one or more analytes to be consumed by one or more cells. A medium can have one or more analytes generated by one or more cells. A medium can also have at the same time one or more analytes to be consumed by one or more cells and one or more analytes generated by one or more cells.

A “gene” is a sequence of nucleotides which code for a functional polypeptide. For the purposes of the invention a gene includes an mRNA sequence which may be found in the cell. For example, measuring gene expression levels according to the invention may correspond to measuring mRNA levels. “Genomic sequences” are the total set of genes in a organism. The term “genome” denotes the coding sequences of the total genome.

The following is a list of notations that may be used in this specification:

Glc, Glucose

Lac, Lactate

CO₂, Carbon Dioxide

O₂, Oxygen

G6P, Glucose-6-phosphate

GAP, Glyceraldehyde-3-phosphate

Pyr, Pyruvate

AcCoA, Acetyl Coenzyme A

α-KG, α-Ketoglutarate

SuCoA, Succinyl Coenzyme A

Fum, Fumarate

OAA, Oxaloacetate

NAD(P)H, Nicotinamide Adenine Dinucleotide and NADPH

FADH₂, Flavin Adenine Dinucleotide

ATP, Adenosine Triphosphate

Ala, Alanine

Arg, Arginine

Asn, Asparagine

Asp, Aspartate

Cys, Cysteine

Gln, Glutamine

Glu, Glutamate

Gly, Glycine

His, Histidine

Ile, Isoleucine

Leu, Leucine

Lys, Lysine

Met, Methionine

Phe, Phenylalanine

Pro, Proline

Ser, Serine

Thr, Threonine

Trp, Tryptophan

Tyr, Tyrosine

Val, Valine

θᵢ_{ᵢ}^{cm}, Stoichiometric coefficient for amino acid i in cellular proteins (mmol/mmol protein)

θᵢ_{ᵢ}^{p}, Stoichiometric coefficient for amino acid i in monoclonal antibody (mmol/mmol MAb)

HTMS, High-Throughput Metabolic Screening.

### Overview of the Invention

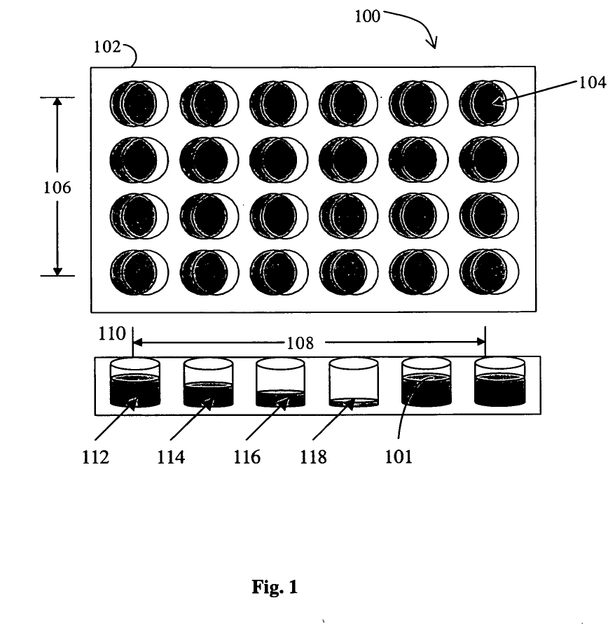

In one aspect, the present invention relates to a system and methods for metabolic screening of cells using well plates. In one embodiment as shown in FIG. 1, a well plate 100 has a body portion 102. The body portion 102 defines a plurality of wells 104, which is arranged in an array having a particular number of rows 106 and columns 108 such that the total number of the plurality of wells L=number of rows 106×columns 108. Well plate 100 can be utilized as an initial culture plate.

In an exemplary operation, a desired initial volume v₀ of sample 101, which is less than 1,000 μL, resides in each well 104. The initial volume, v₀, is represented by 112. Other initial volume may also be chosen. Subsequent to the removal of the first sample, v₁, a volume v₂ remains, shown as 114. After a desired time interval, if a second sample, v₃, is removed, a volume v₄ would remain, shown as 116. If a third sample, v₅, is then removed, a volume v₆ would remain, shown as 118.

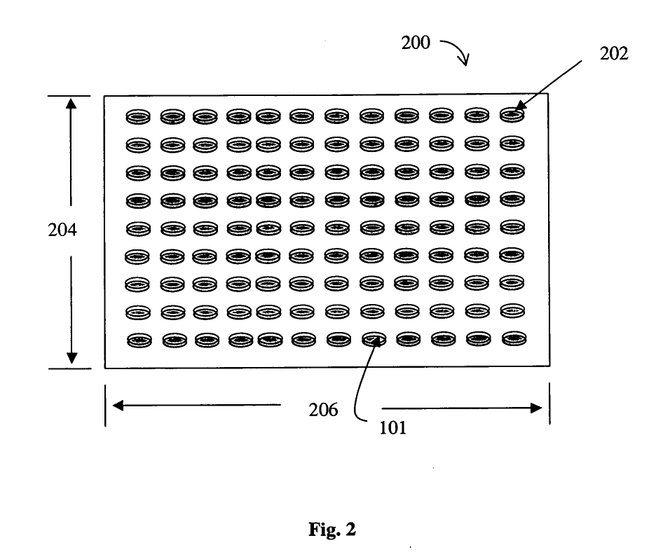

Referring now to FIG. 2, a second plate 200 such as a microtiter plate as shown has a plurality of wells 202. The second plate 200 can be used for the metabolite assays. The second plate 200 has a particular number of rows 204 and columns 206 such that when multiplied, results in the plurality, of wells, M. Each well, 202, as shown, may contain part of the sample 101 from the initial culture plate 100 containing L wells and a particular volume of reagent and enzyme for the assay of interest.

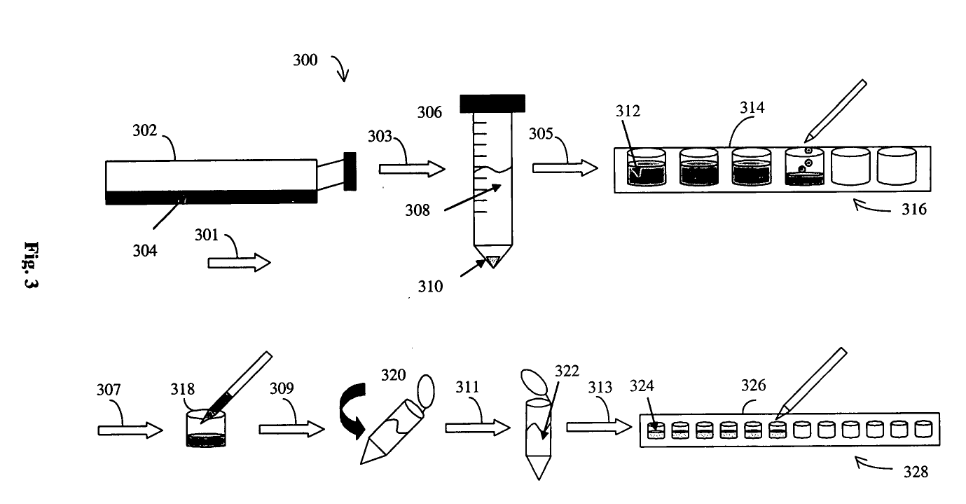

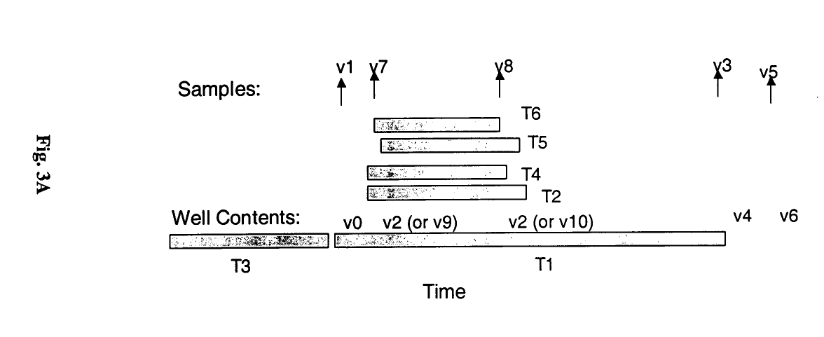

Well plates 100 and 200 can be utilized to perform metabolic screening of cells. In one embodiment, a process 300 for metabolic screening of cells is shown in FIG. 3. At step 301, the process begins with preparing a culture of cells 304 in a culture flask 302. A parent culture 304 is prepared in the form of a solution of medium and cells, where centrifuging and changing medium are conducted as needed to achieve a desired test environment and a desired concentration of cells.

After the culture 304 has grown to a desired density, at step 303, a volume of the culture 304 is sampled and centrifuged in tube 306 to form a pellet of cells 310. The pellet of cells 310 is then resuspended in a volume of control or test medium 308 in tube 306 to be seeded into a first plate 314 having of wells at 305. A side view of the plate 314 is depicted in 316. The initial volume 312 in the well plate is v₀.

At the initial time point, i.e. at step 307, a volume v₁ is sampled from each well 318. At step 309, the cells in the sample are removed via centrifugation 320, and at step 311 the supernatant is collected and transferred to an additional eppendorf tube 322 for immediate use or storage. The steps 307, 309, 311 are repeated after a desired time interval, during which the initial plate 314 containing control and test cultures is incubated. The incubation can be done in an incubator with proper temperature, humidity, and gas phase carbon dioxide control. At 313, metabolites are assayed 324, 326 in a second plate 328 containing L wells, of which a side view is shown.

FIG. 3A is an overview and timeline for the metabolic screening method as shown in FIG. 3 and discussed above. In FIG. 3A, T₁ represents the incubation time that is used between sampling first (v₁) and third (v₃) volumes from one more wells of the first plate. Metabolites such as glucose, lactate, amino acids, and biopharmaceutical are typically assessed over time interval T₁, by analyzing volumes v₁ and v₃ in separate assays. T₂ represents a period of time within T₁ during which the contents of each well during that time period (with volume v2 or v9 or v10) may be monitored two or more times directly by sampling or indirectly using spectroscopy. pH and oxygen may be monitored by spectroscopy or fluorescence during time periods T₄ and T₅, respectively, both within T₂. Total CO₂ (as dissolved CO₂ and bicarbonate) may be monitored by sampling immediately (volume v₇) before and immediately after (volume v₈) a period of time T₆, within T₂, and leaving volumes v₉ and v₁₀ remaining afterwards. T₄, T₅, and T₆ may overlap or coincide exactly, but all are less than or equal to T₂, and T₂ is less than or equal to T₁. One or more wells of the first plate may be sealed during T₄, T₅, T₆, and/or T₂. The entire monitoring period of time T₁ may be preceded by a period of time T₃, during which cells adapt to the new medium, grow, and attach (if applicable). Cell number and culture viability are typically assessed in volume v₅ before T₁ (or T₃) in the parent culture from one or more wells of the first plate after T₁. Volume v6 may be zero or may be used, if not zero, for other cell and molecular biology assays.

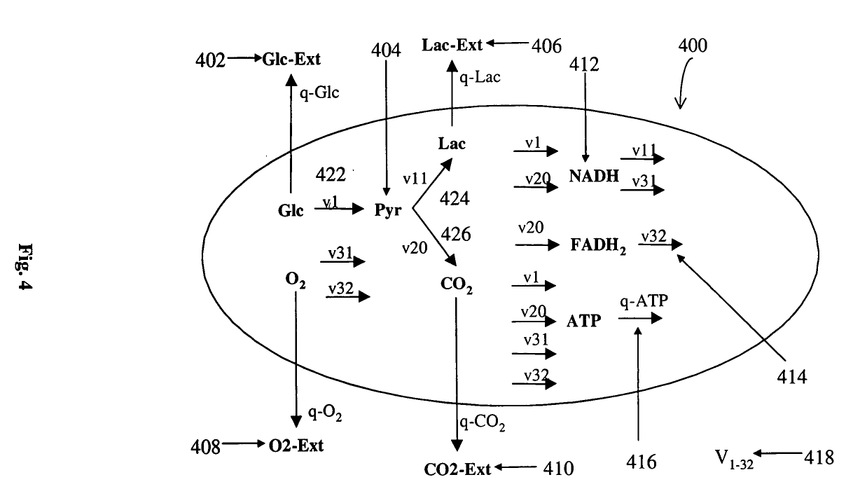

Once raw data of metabolites are measured, metabolic rates can be obtained according to one embodiment of the present invention. Specifically, a first metabolic network 400 is shown in FIG. 4. The first metabolic network 400 can be termed as a “simple metabolic network,” which in no way should limit the scope of the present invention. The first metabolic network 400 provides a model describing four major metabolic pathways that generate energy: glycolysis 422, reduction of pyruvate 404 to lactate 424, the TCA cycle 426, and oxidative phosphorylation 408, 412, and 414. The model includes ten total fluxes 418 represented by vₙ, where n is an integer in the range of 1 to 32. Measured rates for glucose 402, lactate 406, CO₂ 410, and/or O₂ 408 are used to calculate unknown intracellular fluxes as well as estimate ATP generation 416. Measuring all four rates provides redundant measurements that can be used to calculate the consistency of the measurements with the model. Table I illustrates the reaction set that corresponds to the network 400 given in FIG. 4, where reaction 502 represents overall glycolysis, reaction 504 is the reduction of pyruvate to lactate, reaction 506 represents the TCA cycle, and reactions 508 and 510 describe oxidative phosphorylation.

TABLE I

| | | | |
|---|---|---|---|
| 502 | Glc + 2NAD⁺ + 2ADP + 2Pᵢ | → | 2Pyr + 2NADH + 2ATP ++ |
| | | | 2H₂0 + 2H⁺ |
| 504 | Pyr + NADH + H⁺ | → | Lactate + NAD⁺ |
| 506 | Pyr + 4NAD⁺ + FAD + ADP + | → | 3CO₂ + 4NADH + |
| | 3H₂O + Pᵢ | | FADH₂ + ATP + 4H⁺ |
| 508 | 0.5O₂ + 2.5ADP + 2.5Pᵢ + | → | 2.5ATP + NAD⁺ + 3.5H₂0 |
| | NADH + 3.5H⁺ | | |
| 510 | 0.5O₂ + 1.5ADP + 1.5Pᵢ + | → | 1.5ATP + FAD + 2.5H₂O |
| | FADH₂ + 1.5H⁺ | | |

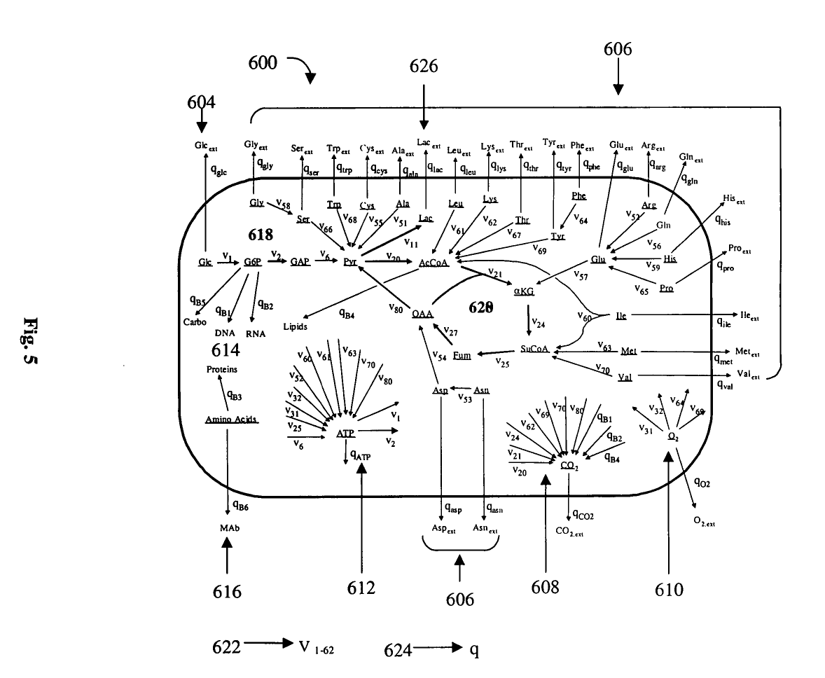

Moreover, a second metabolic network 600 according to one embodiment of the present invention is shown in FIG. 5. The metabolic network 600 includes fluxes 622 represented by vₙ, where n=1, . . . , 62. In other words, the metabolic network 600 has 62 fluxes 622. The metabolic network 600 can be termed as a “detailed metabolic network,” which in no way should limit the scope of the present invention. Similar to the 10-flux network model 400, the detailed model 600 describes glycolysis 604, 618, reduction of pyruvate to lactate 626, the TCA cycle 608, 628, and oxidative phosphorylation 610. Additionally, amino acid production/utilization 606 and synthesis of biomolecules 614 are included. The detailed network 600 incorporates 20 amino acids in the energy network 612, includes 6 fluxes for biosynthesis 614, and accounts for the demand of amino acids and other metabolites when calculating rates that contribute to energy production 612. The simplified network 400 uses measurements of glucose, lactate, oxygen, and CO₂ rates, while the detailed network 600 uses measurements of 31 rates, as well as the use of a stoichiometric model for the cell, to calculate the ATP generation 612. q 624 represents specific rate of production defined positive or consumption defined negative of extracellular metabolites, ATP, biomolecules, and particularly the production of the monoclonal antibody 616. In either case, model calculations and data analysis can be done in a computer.

Table II illustrates the reaction set that corresponds to the network model given in FIG. 600. Reactions 702 through 706 describe glycolysis, reaction 708 is the reduction of pyruvate to lactate, reactions 710 through 718 describe the TCA cycle, reactions 720 and 722 represent oxidative phosphorylation, reactions 726 through 764 depict amino acid metabolism, and reactions 766 through 776 illustrate biosynthesis and monoclonal antibody production.

TABLE II

| | | | |
|---|---|---|---|
| 702 | Glucose + ATP | → | G6P + ADP + H⁺ |
| 704 | G6P + ATP | → | 2GAP + ADP + H⁺ |
| 706 | GAP + NAD⁺ + 2ADP + Pᵢ | → | Pyr + 2ATP + NADH + H₂O |
| 708 | Pyr + NADH + H⁺ | → | Lactate + NAD⁺ |
| 710 | Pyr + NAD⁺ + CoA | → | AcCoA + CO₂ + NADH + H⁺ |
| 712 | AcCoA + OAA + NAD⁺ + H₂O | → | α-KG + CO₂ + NADH + CoA + H⁺ |
| 714 | α-KG + CoA + NAD⁺ | → | SuCoA + CO₂ + NADH + H⁺ |
| 716 | SuCoA + FAD + H₂O + Pᵢ + GDP | → | Fum + FADH₂ + CoA + GTP |
| 718 | Fum + NAD⁺ + H₂0 | → | OAA + NADH + H⁺ |
| 720 | 0.5O₂ + 2.5ADP + 2.5Pᵢ + NADH + | → | 2.5ATP + NAD⁺ + 3.5H₂0 |
| | 3.5H⁺ | | |
| 722 | 0.5O₂ + 1.5ADP + 1.5Pᵢ + FADH₂ + | → | 1.5ATP + FAD + 2.5H₂O |
| | 1.5H⁺ | | |
| 724 | OAA + ADP + Pᵢ + 2H⁺ | → | Pyr + CO₂ + ATP + H₂O |
| 726 | Ala + α-KG | → | Pyr + Glu |
| 728 | Arg + 2NADP⁺ + ADP + Pi + | → | Glu + 2NADPH + ATP + 3NH₃ |
| | 2H₂O | | |
| 730 | Asn + H₂O | → | Asp + NH₃ |
| 732 | Asp + α-KG | → | OAA + Glu |
| 734 | Cys + NADP⁺ + H₂0 | → | Pyr + NADPH + NH₃ |
| 736 | Gln + H₂O | → | Glu + NH₄⁺ |
| 738 | Glu + NADP⁺ + H₂0 | → | α-KG + NADPH + NH₃ |
| 740 | Gly + H₂O + MTHF | → | Ser + THF |
| 742 | His + 2H₂O + THF | → | Glu + NH3 + MTHF |
| 744 | Ile + 2CoA + ATP + FAD + | → | AcCoA + SuCoA + ADP + NH₄⁺ + FADH₂ + |
| | 3NAD⁺ | | 3NADH |
| 746 | Leu + FAD + 2NAD⁺ + 2ATP | → | 3AcCoA + FADH₂ + 2NADH + |
| | | | 2ADP + NH₄⁺ |
| 748 | Lys + CoA + FAD + 5NAD⁺ | → | 2AcCOA + 2CO₂ + 2NH₄⁺ + FADH₂ + |
| | | | 5NADH |
| 750 | Met + ATP + CoA + H₂O + H⁺ | → | SuCoA + ADP + Pi + NH₃ + Sulfide |
| 752 | Phe + O₂ + NADH + H⁺ | → | Tyr + NAD⁺ + H₂O |
| 754 | Pro + 2NADP⁺ + H₂O | → | Glu + 2NADPH |
| 756 | Ser + H₂0 | → | Pyr + NH₃ + H₂0 |
| 758 | Thr + NAD⁺ + CoA | → | AcCoA + Gly + NADH + H⁺ |
| 760 | Trp + H₂O | → | Pyr + Indole + NH₃ |
| 762 | Tyr + NADP⁺ + 2O₂ + H₂0 + | → | 2AcCoA + Fum + NADPH + CO₂ + NH₃ |
| | 2CoA | | |
| 764 | Val + CoA + ATP + FAD + 4NAD⁺ | → | SuCoA + ADP + CO₂ + NH₄⁺ + FADH₂ + |
| | | | 4NADH |
| 766 | G6P + 1.25Asp + 2Gln + 0.5Gly + | → | DNA + 2Glu + 0.75Fum + |
| | 7.5ATP + 1.25NADP⁺ + H₂O + 1-C | | 1.25NADPH + H⁺ + CO₂ |
| 768 | G6P + 1.25Asp + 2Gln + 0.5Gly + | → | RNA + 2Glu + 0.75Fum + CO₂ + |
| | 7.5ATP + H₂O + 1-C | | 2.5NADH + H⁺ |
| 770 | Σθᵢ_{₁}^{cm} AAᵢ + 31.3ATP | → | Proteins |
| 772 | 0.24Glu + 18.5AcCoA + 0.24 G6P + | → | Lipids + 0.24α-KG + 0.47NADH + CO₂ |
| | 0.5GAP + 17.5ATP + 24.5NADPH | | |
| 774 | G6P + ATP Glycogenₙ + H₂0 | → | Glycogenₙ₊₁ + ADP + 2Pᵢ |
| 776 | Σθᵢ_{₁}^{p} AAᵢ + 31.3ATP | → | MAb |

Exemplary system and methods according to the embodiments of the present invention are given below. Note that titles or subtitles may be used in the examples for convenience of a reader, which in no way should limit the scope of the invention.

### EXAMPLES

### Example 1

### Metabolic Screening of Mammalian Cell Cultures Using Well-Plates

Introduction

In line with the established biological paradigms, metabolism may be considered to lie at some sort of median between genetics and cell physiology. With the insurgence of proteomics as a robust tool for biological engineers, there is a growing need to quantify the specific relationship between a cell's genotype and phenotype. Metabolic pathway analysis may be the answer in providing a connection between the vast amounts of genomic and proteomic data being generated from current array technologies. Modeling cellular metabolism in conjunction with a specific genotype can be an extraordinary tool in optimizing growth patterns, therapeutic protein production, and cellular environments and targeting proteins for novel drug development. Observing metabolic patterns in mammalian cells under varying environmental and genetic conditions documents the changing trends in specific biochemical pathways as it relates to cell physiology. However, the measurement, quantification, and cataloging of metabolic pathways is in its infancy compared to throughput and application of genomic methods. Metabolic rates of mammalian cells in culture have been measured predominantly in macro-scale systems (such as T-flasks, spinner-flasks, and bioreactors) operated in batch, fed-batch, or continuous mode. Throughput and replication are low, capital and operating expenses are moderate to high, experiments are time-consuming (days to weeks), and such techniques often require specialized expertise. Additionally, to improve accuracy, measurements are obtained for steady or pseudo-steady state conditions at the expense of insight into metabolic dynamics and regulatory control loops. Thus, today's cellular and metabolic engineer needs a way to more easily, rapidly, and economically tap into the wealth of information metabolism has to offer in order to better understand “genome-physiology connections”. And just as genes and proteins are being databased, metabolic information should be cataloged for the creation of complete models of single cells that would offer researchers the complete genetic and biochemical information that determines cell physiology.

In contrast to metabolic measurements, metabolic network theories and metabolic models are quite developed and ready to complement genomics and proteomics. One methodology in metabolic engineering, metabolic flux analysis, expresses cellular metabolism in the form of sequenced, observable biochemical reactions (pathways) and defines the pathway flux as the rate at which input metabolites are being converted to reaction products. In attempting to create metabolic models for steady state as well as transient conditions, the objective is to describe phenotype in the terms of metabolic fluxes. While there still exist limitations in metabolic models, such as a lack of accounting for metabolic regulation points and reversible pathways, researchers have been able to develop and use various network models to more comprehensively quantify metabolism of mammalian using measured metabolic rates.

One advantage offered by the present invention is to empower researchers with a preliminary way to start including metabolic measurements along with other genomic and proteomic screens. To accomplish this, we have increased measurement throughput while decreasing model complexity. We first use a theoretical analysis to demonstrate the feasibility of assessing metabolism using a simplified, 10-flux metabolic network requiring a minimum of 2 measurements as compared to a more detailed, 64-flux metabolic network requiring a minimum of 28 measurements. We next describe the modification of standard, well-known methods and assays in cell culture to achieve a composite protocol for more rapidly and more inexpensively determining metabolic rates for use with the simplified network. Finally, we show an example experiment in which HTMS is applied to screen for potential metabolic effects of anti-apoptotic doses of rapamycin. Rapamycin increases resistance to cell death and doubles specific productivity during fed-batch cultivations of our hybridoma cell line by an unknown mechanism. Under a hypothesis that metabolic capacity may be governing death and specific productivity, we used HTMS to estimate specific ATP production from and flux distributions among the major energy pathways for control and rapamycin-fed cultures. The overall result of using higher-throughput methods in conjunction with a simplified network that demands fewer measurements for estimation of metabolic capacity is a 20-fold increase in throughput for preliminary metabolic screening and simplified metabolic flux analysis, when compared to the throughput achievable with T-flasks.

### Materials and Methods

Metabolic Network Model 1.

Metabolic network Model 1 (shown in FIG. 5) is a moderately detailed, metabolic flux analysis model that we used as a basis for comparison of results estimated from the same rate data but using the simpler model, Model 2. It is an adaptation of a network used previously for CHO and hybridoma cells, but may be used with other cultured mammalian cells with similar growth properties. In the model, there are 32 intracellular fluxes and 30 fluxes for transport rates and biosynthesis rates that can be measured or estimated. 35 species constitute nodes for pseudo-steady state balances. 12 of the intracellular fluxes are overall biochemical reactions (simplified by lumping serial pathways together) representing four major metabolic pathways involved in energy production: glycolysis, reduction of pyruvate to lactate, the TCA cycle, and oxidative phosphorylation. The pentose phosphate pathway is represented only for the generation of DNA and RNA in biosynthesis reactions, since the solution with its inclusion in full is not possible by material balances alone. 20 overall biochemical reactions, which incorporate the stoichiometries of several to many individual reactions, represent the degradation of amino acids to intermediates in the energy pathways. Degradation, rather than biosynthesis, reactions were chosen in cases where more than one pathway was possible and all pathways were verified as feasible for Mus musculus on the KEGG website (http://www.genome.ad.jp/kegg/kegg2.html).

6 biosynthetic reactions for DNA, RNA Protein, Lipid, Carbohydrates, and MAb are used to account for consumption of pathway intermediates and amino acids for biosynthesis. The rates for each of the biosynthesis reactions were calculated from the dilution rate and cell density of the steady state times the fraction of cell weight and the average molecular weight of the representative biomass species. In the case of protein and MAb, the stoichiometry of the reaction was scaled down by specifying a nominal molecular weight of 1000 for the proteins or MAb in order to reduce that condition number of the stoichiometric matrix. Conversely, the reaction rate was scaled up by the same factor to achieve the same overall mass balance. It was verified that such scaling did not alter the values of the estimated fluxes. Finally, a growth rate constraint equation, specifying that the sum of the mass rates per cell mass for the 5 biosynthesis reactions for cellular macromolecules is equal to the growth rate, was included as an additional redundant equation in the solution. This equation was added to prevent undo alteration of the biosynthesis rate specifications during the least-squared fitting of the solution vector of calculated fluxes and was successful in allowing biosynthetic rates to be adjusted and distributed within the given growth rate measurement. This was done instead of arbitrarily specifying zero noise on the biosynthesis rates that clearly have large noise since they were estimated from literature values and simplified biosynthetic pathways (not shown). The coefficients of this equation were scaled down by a factor of 10⁹ to maintain a reasonable condition number.

24 transport fluxes are formally defined for each measured metabolite, and each rate is defined with a positive sign for production. Thus, for example, the glucose transport flux points outward and is negative since glucose is consumed. Each extracellular metabolite is linked to its intracellular counterpart metabolite pool, a representation that is presumably accurate for most metabolites except glucose, which is immediately converted to glucose-6-phosphate and does not essentially have an intracellular pool. The conventions used in this model allow for the incorporation of measured metabolic rate data and their noise estimates directly from experimental data sets, without further data reduction or changes in signs.

Energy in the forms of NAD(P)H, FADH₂, GTP, and ATP produced from all reactions, except biosynthesis, are converted and balanced into an overall ATP production flux. NAD(P)H and FADH₂ are balanced with zero net production or consumption, with reactions 31 and 32 for oxidative phosphorylation being used to generate ATP. The P:O ratio is specified as 2.5, which comes from a P:O of 3 minus the energy needed for transporting ATP to the cytosol. Furthermore, NADH formed in the cytoplasm is assumed to yield the same amount of ATP as that formed in the mitochondria, implicitly specifying the use of the malate-aspartate shuttle for their electrons. Finally, GTP made during TCA cycle is considered to be an equivalent of ATP, and being concomitantly produced with FADH₂ makes the P:O ratio for both reactions 31 and 32 equal to 2.5, as pointed out previously. In this way, the net energy produced by the activity of pathways modeled is indicated by the value of q_{ATP}, one of the estimated fluxes. q_{ATP} serves as a principle output for either model.

Ammonia, water, hydrogen ions, Enzyme CoA, phosphates, and other molecules are neither measured nor balanced in this model. Overall for Model 1, the reactions used and balances made result in a stoichiometric matrix of dimension 36×64 (not shown), which is of full rank and has a condition number of 80.5. In addition, the specification of 31 fluxes (for 24 metabolic quotients, 6 biosynthesis rates, and 1 growth rate) yields a system of linear equations that is overspecified by three degrees.

METABOLIC NETWORK 2. A simplified, 10-flux metabolic network (FIG. 4) was devised and analyzed for use in generating fluxes and q_{ATP} from a subset of the measured metabolic rates needed for the detailed network. Model 2 still represents four major metabolic pathways that generate energy, but was simplified by excluding all transport and biosynthesis fluxes that were less than 10% than the largest flux estimated from Model 1. The only remaining measured fluxes were for glucose, lactate, CO₂, and O₂; glutamine was the next largest flux, but only 3-6% of the largest flux in the data sets. This same network also follows from the independent creation of a metabolic network for energy production where amino acids and biosynthesis are neglected a priori. With smaller measured fluxes neglected, lumping serial reactions together and eliminating redundancies simplified the more detailed network considerably.

Model 2 incorporates only four measured rates for glucose, lactate, carbon dioxide, and oxygen, and just as for Model 1, provides an estimate of ATP production as a principle output. As 8 balances on pathway intermediates (Glc, Pyr, Lac, CO₂, O₂, NADH, FADH₂, and ATP) relate the 10 fluxes, specifying just two measured rates yields an exactly determined system of linear equations. Indeed, many researchers have used lactate and oxygen measurements to calculate ATP production, and this simplified network exactly mirrors their calculations once the P:O ratio and malate-asparate shuttle specifications are made identical. From Model 2, it is clear that other permutations of dual measurements are feasible, and we exploit this fact by developing glucose and lactate measurements first, since they were easier to obtain from well-plates. Model 2 also allows for using redundant measurements. Measuring oxygen and/or carbon dioxide in addition to glucose and lactate would provide a system that is overdetermined by one or two degrees and which can therefore be used for consistency testing and gross error detection.

Overall for Model 2, the reactions used and balances made result in a stoichiometric matrix of dimension 8×10 (not shown), which is of full rank and has a condition number of 7.6. In addition, the specification of 4 fluxes (for 4 metabolic quotients) yields a system of linear equations that is overspecified by two degrees.

Metabolic Rate Data for Comparison of Models

Raw data used for generating metabolic rates for 24 substrate or product metabolites (glucose, lactate, oxygen, carbon dioxide, and 20 amino acids) were obtained from previously documented steady state chemostat hybridoma culture experiments. These series of steady states spanned a broad range of dilution rates (Steady states A, B, C, and D were obtained sequentially for dilution rates of 0.04, 0.03, 0.02, and 0.01 hr⁻¹, respectively), and a multiple metabolic steady state was observed upon return to the high dilution rate of 0.04 hr⁻¹. For this analysis, measured metabolic rates and estimates of their errors were recalculated (using the method explained below) in order to estimate the errors on the rates based on uniform values for noise on prime variables and generate net amino acid production rates, since only the rates for energy were shown previously. Moreover, 6 rates for biosynthesis of cellular macromolecules (DNA, RNA, Proteins, Lipids, and Carbohydrates) and MAb product (as described in the section for Model 1) were used instead of rates for individual pathway intermediates used formerly. All 31 measured rates (for glucose, lactate, carbon dioxide, oxygen, 20 amino acids, 6 biosynthesis, and growth rate) were used as inputs for Model 1, whereas just 4 measured rates (glucose, lactate, carbon dioxide, and oxygen) were used for Model 2.

Metabolic Flux Analysis

Estimates for unmeasured fluxes (the fluxes to be calculated) in Models 1 and 2 were determined using the Tsai-Lee method as known to people skilled in the art. This method determines calculated fluxes that are least-square fits of the measured fluxes with the overdetermined model balance equations. In this method, errors on balance equations are minimized and estimates for fluxes corresponding to measured rates are slightly adjusted within the range of their noise. Thus, the output of the calculation is a set of estimates for both measured and calculated fluxes. Each data set was tested for statistical consistency with Models 1 and 2 using the consistency test function as known to people skilled in the art.

Measured fluxes for 24 metabolites and 6 biosynthesis reactions were calculated from a set of prime variables that are independent and directly measured from the bioreactor system, or, in the case of biosynthetic reactions, estimated from a model with literature values. Uniform values for random errors (noise) on prime variables were used across all five steady states. Such noise was ascertained from the standard deviation associated with each raw measurement. As known to people skilled in the art, estimates for errors on measured rates were calculated from the first order partial derivatives (the sensitivities to each prime variable) of the measurement rate formula. The sensitivities were also used to generate an approximation of a variance-covariance matrix that is used in the calculation of flux estimates and in the consistency test.

Cell Culture

The cell line used was a murine hybridoma (ATCC CRL-1606) that secretes an immunoglubulin IgG against human fibronectin. During maintenance, the hybridomas were cultivated in a serum-free, hydrolysate-free IMDM formulation, comprised of glutamine-free IMDM basal medium, 4.0 mM glutamine, 10 mg/L insulin, 5 mg/L holo-transferrin, 2.44 μL/L 2-aminoethanol, 3.5 μL/L 2-mercaptoethanol, and 10 U/ml penicillin-10 □g/ml streptomycin. For the HTMS experiments, the cells were cultivated in RPMI 1640 (Sigma Chemical Co.) supplemented with 2 g/L sodium bicarbonate, 4.0 mM D-glucose, 10 mg/L insulin, 5 mg/L holo-transferrin, 2.44 μL/L 2-aminoethanol, 3.5 μL/L 2-mercaptoethanol, and 10 U/ml penicillin-10 μg/ml streptomycin. Experiments in which rapamycin (Sigma Chemical Co.) was added were given the appropriate amount of a rapamycin stock solution, which was made at 500 μM in ethanol and stored as 200 μL aliquots at −70° C. for up to six months. Controls were given an equivalent amount of ethanol.

High-Throughput Metabolic Screening

HTMS experiments were performed in 24-well plates (BD Falcon). Hybridoma cells were cultivated in T-175 cm² flasks, centrifuged at 200 g for 10 minutes, and resuspended in control or test medium. The cells were then seeded into a 24-well plate at a density of 1.75×10⁶ cells/mL and incubated for four hours at 37° C., 95% humidity, and 10% CO₂. Samples for initial concentrations of glucose and lactate were removed from the culture used to seed the well plate. Samples from each well were removed after 4 hours, centrifuged, and supernatants were stored at −20° C. for analysis at a later time. That evaporation was not occurring significantly was determined by analyzing samples taken initially and after 4 hours from a well-plate loaded with test medium but without cells (data not shown). The rapamycin concentrations for the metabolic screening of rapamycin ranged from 50 to 1000 nM.

Metabolite Assays

Triplicate measurements of glucose and lactate concentrations for each well of a 24-well plate at initial and final time points were conducted in separate 96-well plates, using well-known enzymatic assays, which we reformulated for use in 96-well plates. All absorbance measurements were performed on a □-Quant UV/vis plate reader (Bio-tek).

Calculation of Metabolic Rates from HTMS Experiment

Rates for each well of the HTMS experiment were calculated as the change in concentration divided by the cell density of the seed culture and the time interval of 4 hours. Average rates for control and rapamycin HTMS cultures were taken as the average and standard deviation from 4 wells. The statistical treatment of data was accomplished using Microsoft Excel.

Results

SIMPLIFIED METABOLIC NETWORK (MODEL 2) PROVIDES ATP PRODUCTION RATES AND FLUX DISTRIBUTIONS SIMILAR TO DETAILED NETWORK (MODEL 1). To investigate the prospect of using Model 2 to estimate ATP production and changes in metabolism from measured rates, we compared ATP production, percent ATP from glycolysis and TCA cycle, percent carbon flux through glycolysis and TCA cycle, and lactate-glucose ratios that were obtained from flux estimates generated by either model for a series of steady state data sets. For Model 2, we specified four metabolic rates (glucose, lactate, carbon dioxide, and oxygen), while for Model 1 we specified 24 metabolic rates (the four used for Model 2 plus all 20 amino acids), 6 biosynthesis rates (DNA, RNA, proteins, lipids, carbohydrates, and MAb), and the growth rate.

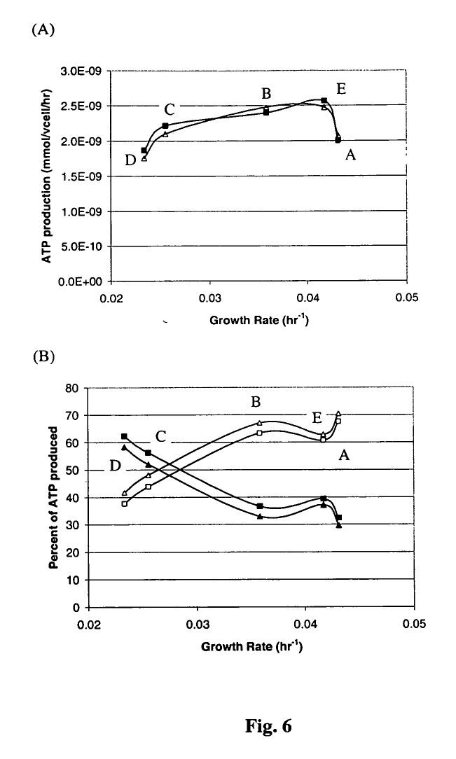

With regard to energy production, the ATP production rates obtained from Model 2 were found to generally reflect those obtained from Model 1 (FIG. 6A). Model 2 slightly overestimates ATP production for steady state A and B, and slightly underestimates them for C, D, and E. Nevertheless, both models demonstrate a similar trend that steady states B and E have higher ATP production rates than A, C, and D. Model 2 overestimates the amount of ATP produced by glycolysis relative to that produced by the TCA cycle by 2-5% (on the absolute scale) as compared to Model 1 (FIG. 6B). Even still, both models show a similar shift in the source of ATP production towards TCA cycle for lower growth rates.

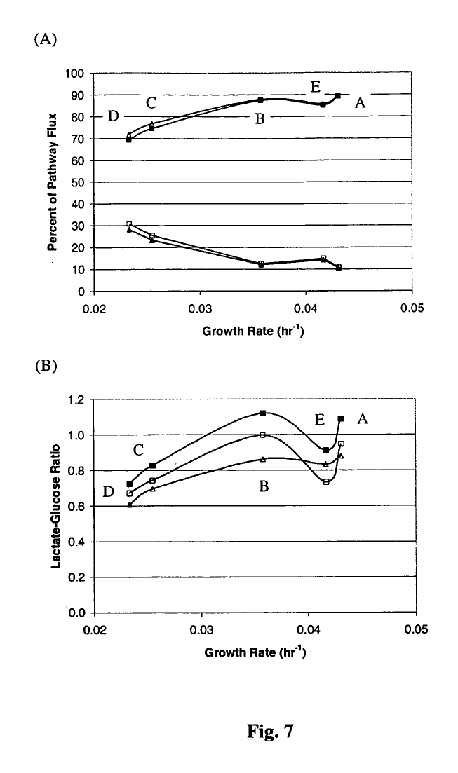

Besides ATP production, we verified the ability of Model 2 to reasonably relate changes in flux distribution. Each model provided very similar estimates for the amount of 6-carbon flux through glycolysis and the TCA cycle (FIG. 7A). From either model, TCA cycle activity increased from about 10% to 30% as the growth rate decreased. Another comparison of flux distributions from Models 1 and 2 is that for lactate-glucose ratios (FIG. 7B). Here, both models differ notably from the direct calculation of lactate-glucose ratio from measured rates not using any model. Model 1 overestimates the ratio, while Model 2 underestimates the ratio for all data sets except steady state E.

Finally, Model 2 provided the same outcomes for statistical consistency between measurement data sets and model as were obtained with Model 1 (Table 3). Steady states A, B, C, and D were found to be consistent within a chi-square confidence level of 90%, while steady state E (the multiple steady state) was not.

MODIFIED ENZYMATIC ASSAYS AND CELL CULTURE PROCEDURES ENABLE HIGHER-THROUGHPUT METABOLIC SCREENING (HTMS) WITH WELL-PLATES. To complement the simplicity of Model 2 that requires just 2-4 metabolic rate measurements, we devised a relatively simple experimental protocol (as compared to those with T-flasks and bioreactors) that capitalizes on well-plate technology for increased throughput. In essence, we use 24-well plates to culture 24 independent cultures at a time. Each culture is sampled initially and several hours later, and the 24 samples are analyzed for glucose and lactate, each in triplicate, within separate 96-well plates.

Our ability to determine average metabolic rates from each 400 μl, 4-hr, batch culture on a 24-well plate with quite good precision was made possible through several key adaptations of conventional cell culture methods. First, the test medium, which is the medium used in the well-plate as opposed to growth medium used to culture cells in the long-term, was made with lower glucose concentration (4.0 mM) and remained serum-free. Second, the assays for glucose and lactate were reformulated to span calibration curves ranging from 0-5 mM for glucose and 0-3 mM for lactate. Third, we forgo deproteination of samples during glucose and lactate assays since it may be neglected when the protein content of the samples is sufficiently low. Yet, even with expected low protein levels, we test non-deproteinated vs. deproteinated samples for each new system to ensure no negative interaction with the enzymatic assays (data not shown). Together, these changes allow us to use samples without diluting them. Thus, a sample taken from a well can be used directly in the enzymatic assay, avoiding the labor and experimental noise associated with deproteinating and diluting. The net result is a much-reduced noise associated with measured concentrations, as compared to the standard, cuvette-based method. With such precision, a 1.0 mM change in glucose in a single well has experimental error of 14% or less, and a 1.0 mM change in lactate has experimental error of 4% or less.

HIGH THROUHGPUT METABOLIC SCREENING OF RAPAMYCIN TREATED HYBRIDOMAS. As a demonstration of HTMS, we provide the results of an experiment in which we screened the metabolism of hybridomas given various concentrations of rapamycin. Previously, 100 nM rapamycin was determined to be optimal for production of monoclonal antibodies from our cell line, as it delayed cell death for approximately one day and doubled the specific productivity of MAb. In this experiment, we screened for metabolic differences between control cultures and those with 50, 100, 250, 500, or 1000 nM rapamycin. Each concentration was replicated 4 times for a total of 24 simultaneous cultures on one plate. Metabolism was measured over a 4-hr time interval using HTMS.

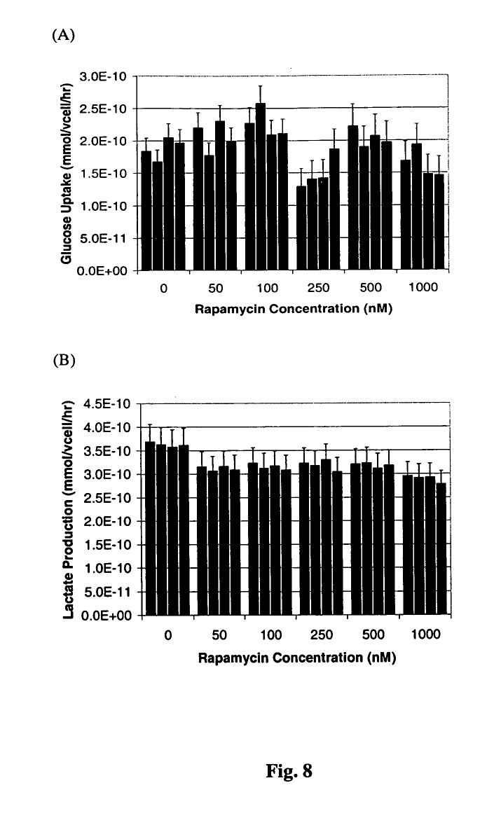

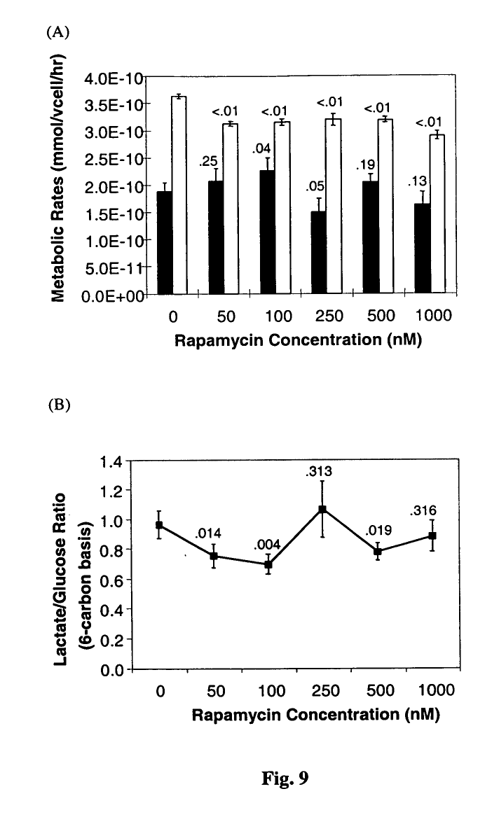

Metabolic rates from individual wells of the 24-well-plate highlight the throughput of HTMS (24 metabolic data sets obtained in 4 hours), the magnitude of the estimated noise on the rates, and the well-well variation within and between concentration groups (FIG. 8). For individual wells, error bar estimates range from approximately 10-20% for glucose rates and approximately 10% for lactate rates. Within groups, none of the rates are statistical outliers, though the glucose rates showed more intra-group variation than the lactate rates. Capitalizing on the multi-replicate nature of HTMS, we calculated average rates and standard deviations from well cultures given the same rapamycin concentration (FIG. 9A). Two-tailed t-tests assuming unequal variance show that 100 and 250 nM cultures had significantly different glucose rates (to a 95% confidence level) compared to the control culture, while all test cultures were significantly different (better than 99% confidence) with regard to lactate production. Lactate-to-glucose ratios were also computed for each well and used to generate the average L/G ratio and standard deviation for each concentration group (FIG. 9B). As for measured rates, t-tests were used to determine that cultures with 50, 100, and 500 nM rapamycin had significantly different ratios compared to the control.

Metabolic flux analysis using Model 2 was next used in further analyzing the metabolic data sets. Glucose and lactate rates for each well were used as inputs for determined solutions of Model 2. This provided 24 sets of fluxes for v₁, v₁₁, v₂₀, v₃₁, v₃₂, and q_{ATP}. However, five of 24 data sets (2 of the control wells and 3 of the 250 nM wells) resulted in negative fluxes for v₂₀, v₃₁, and v₃₂ as their L/G ratio was greater than 1.00 on a 6-carbon basis. Without further recourse, the 5 wells that yielded negative fluxes were excluded from further calculations regarding energy production and flux distribution. Because of this, statistical significance was limited to a comparison against just the two remaining control wells, and no significance test could be done for data from the solitary 250 nM well.

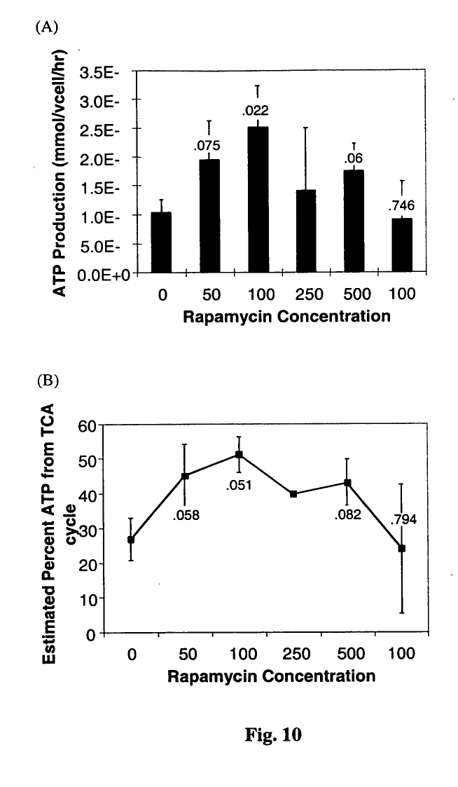

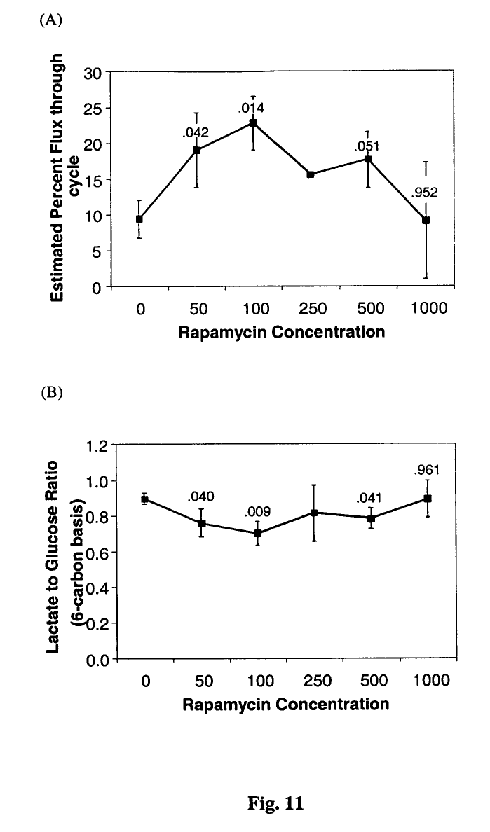

A plot of average ATP production for each group shows that ATP production was either similar or increased for cultures with rapamycin (FIG. 10A). The increase was greatest for 100 nM cultures, and was statistically significant to the 95% confidence level. A plot of percent ATP from the TCA cycle shows that the increase in ATP production appears to be associated with a metabolic shift towards ATP production from the TCA cycle, but no such shifts were significant at the 95% confidence level (FIG. 10B). Yet, looking at changes in flux distribution (FIG. 11A), it appears that cultures with 50 and 100 nM did exhibit a significant increase in percent flux through the TCA cycle. Additionally, L/G ratios (FIG. 11B) are significantly lower for 50, 100, and 500 nM cultures, a result that mirrors that obtained directly from the measured rates.

### Discussion

Results from the theoretical comparison of Model 1 and 2 demonstrate that Model 2, the simple 10-flux model, can in fact generate metabolic information that is similar to that obtained from a more detailed, 64-flux model. Estimates of ATP production were quite similar, even though Model 2 overestimated ATP generated from glycolysis. Estimates of flux distribution showed similar results for relative amounts of carbon flowing through glycolysis and the TCA cycle, even though lactate-glucose ratios were underestimated compared to Model 1 and the direct calculation. The similarity in ATP production and flux distribution is remarkable. The deviations in percent ATP from glycolysis and lactate-glucose ratios are understandable given the exclusion of other carbon sources; even still, such deviations were systematic and hence such estimates could still relate the same overall changes.

Model 2 is the metabolic flux analysis embodiment of previous simple methods for estimating ATP production. Importantly, Model 2 reproduces (after matching assumptions regarding P:O and NADH from the cytosol) these relationships when it is solved as a determined system of equations after specifying two of the four measurable rates. Model 2 is also the bare minimum for a metabolic flux analysis network, and it defines the most minimal set of measured rates to determine ATP and observe differences in metabolism. While quite simple, Model 2 still allows for incorporation of redundant measures and use of consistency testing, features that may become more important for parallel, micro-scale, and batch experiments not necessarily conducted at steady state.

The comparison of lactate-glucose ratios from Model 1 and the direct calculation in the theoretical analysis highlights the systematic errors that can be inherent in MFA networks and witnessed using a solution method that adjusts experimentally determined rates within their noise. In model 1 calculations, all five of the glucose rates output from the least-square solution are less than their directly measured counterparts (not shown). According to the model, less glucose was consumed during those steady states relative to the amount actually measured. The discrepancy could be due to several reasons: (1) the glucose measurement was systematically high (2) any of the 30 other specified measurements were systematically high or low, (3) the cell weight, cell composition, and/or protein composition used were inaccurate, (3) the reactions used for energy metabolism, amino acid uptake, and/or biosynthesis contain an error, or (4) some combination of the above. In our calculations, we did not alter model parameters in order to achieve better agreement in lactate-glucose ratios and purposefully used the Tsai-Lee solution method to clearly highlight the present discrepancy. The reconciliation of lactate-glucose ratios (and other calculated estimates) from Model 1 with the direct measurements is left to future modeling efforts and experiments.

In addition to comparing the discrepancy between calculated estimates for measured rates and the measured rates themselves, we used the consistency test function to provide an overall statistical assessment of the fit between data sets and models. Such analysis showed that Model 1 and 2 provided the same answers: deviations on mass balances for A, B, C, and D were likely to be due to noise on the measurements, while deviations for E were not likely to be due to noise alone, pointing to some systematic error in model or measurement. The findings that the four measured rates and Model 2 could be consistent and, further, that consistency with Model 2 mirrored that with Model 1 is perhaps quite remarkable. That glucose, lactate, oxygen, and carbon dioxide are the major fluxes in comparison to amino acids and biosynthesis is reflected in our ability to make use of self-consistent network that neglects amino acid uptake and consumption of metabolites for biosynthesis.

In this study, we used the same noise estimates for prime variables for all data sets, making the assumption that the noise on measurements did not vary significantly over the course of measuring the steady states in series. So, the discrepancy amounts to a technicality of how the noise was specified. The use of uniform noise estimates, rather than observed standard deviations, however, should allow for better comparison of consistency test functions from similarly executed experiments, and as such, will employed in the analysis of consistency in HTMS experiments. Besides consistency, steady state E does appear to be different than the other steady states, even when looking just at the lactate-glucose ratios. That steady state E was borderline consistent in a single measurement of a steady state in a bioreactor is perhaps just a curious point. Without replication, the measured rates are what they were. However, an essence of HTMS is that the observance of inconsistency in multi-replicate, multi-parallel well experiments may provide an unprecedented basis for querying the metabolic nature of and underlying mechanism for inconsistencies that are not due to random fluctuation of measurements.

Thus, based on the comparative theoretical analysis with Model 1 (detailed) and Model 2 (simple), we conclude that it should be possible to use Model 2 to obtain estimates ATP production and flux distribution between glycolysis and the TCA cycle. Obviously, the detail offered by more detailed networks is beneficial for pinpointing particular pathways or individual reactions and genetic and enzymatic elements involved in the changes. Yet, for the purpose of preliminary screening of metabolism, we offer that the simplified network may be sufficient to identify factors and conditions or interest.

Experimentally, we have adapted standard laboratory techniques to demonstrate the concept of metabolic screening of mammalian cells with well-plates. The use of well-plates or micro-scale culture is not without precedence. Cell culture researchers have used 6-well plates to investigate growth, cell death, cell cycle, and metabolism as functions of environmental parameters such as glutamine, insulin, and dissolved CO₂. Meanwhile, well-plates (96, 384, or higher) are commonly employed for combinatorial chemistry and biological applications. To our knowledge, our use of well-plates for metabolic screening, and its coupling to a simplified metabolic network model, are novel. We have demonstrated the idea of doing metabolic flux analysis from simultaneous measurements taken from micro-scale cultures.

If metabolism and metabolic engineering is to help integrate genomics and proteomics in relation to defining phenotype, this work takes a step towards increasing the throughput for measurements of metabolic rates. A single researcher might use 8 T-flasks in parallel, sampling every 24 hours in order to sufficiently quantify the concentration changes from low to moderate cell density cultures and standard enzymatic techniques, generating a net of 8 metabolic data sets per 24 hours. In comparison, a single researcher can generate 24 data sets in 4 hours time using HTMS. Neglecting time for preparation and analysis of samples, we roughly estimate the increase to be about 20-fold, triple the data sets in one-sixth the time. The throughput could be even greater in some applications where metabolic screening could focus or replace screening for viability. Furthermore, metabolic screening also offers higher content than viability screening. We monitored glucose and lactate since they were the easiest of the four possible measurements to reduce to practice. Having successfully proven our concept, work is ongoing to devise measurements of oxygen and carbon dioxide in well plates, which would provide redundant rate measurements for use in Model 2 calculations. At the same time, the largest source of variation for metabolic rates now falls predominantly on the accurate estimation of cell density and viability for each well.

Besides improving measurement techniques, we believe that effective metabolic screening in well-plates (or other micro-scale systems) will rely heavily on appropriate design of test media. For a relative screen, as shown for rapamycin herein, the test medium comprised mainly of fresh RPMI was sufficient to observe metabolic differences. Yet, the absolute metabolic rates of the hybridomas were in fact different than the rates for our cell line determined previously in batch, fed-batches, and continuous bioreactors. Indeed, we also used HTMS (with the same, nominal test medium) to track metabolism of cells taken from batch cultures, verifying differences in absolute metabolism between cells in the batch (measured using 24-hr time points) and cells taken from the batch and placed in well-plates (unpublished). To upgrade HTMS from relative screening to quantitative metabolism, the micro-scale environment will have to be designed to represent that of the system being analyzed, whether it is a bioreactor or an in vivo tissue.

The metabolic screen of rapamycin in hybridoma cultures was used to illustrate the methodology of HTMS, as well as query potential metabolic effects of rapamycin on hybridomas. As demonstrated, HTMS enables the use of statistical methods in analyzing differences in data sets for metabolism as a function of some change in experimental conditions. Average of rates from multi-replicates cultures provided much more precise rates. Having measured glucose and lactate rates, the use of Model 2 was limited to wells that exhibited lactate-glucose ratios less than 1.0 on a 6-carbon basis. While negative values for flux v₂₀ (pyruvate leading to CO₂) in Model 2 were not realistic, such a situation corresponds to a net flow of carbon from the TCA cycle to the pyruvate node (flux v₂₀ less than v₈₀), as was found for steady state A and B in Model 1 (data not shown). For determined systems, measurement of glucose or lactate with oxygen or CO₂ would be preferred, whereas measurement of three or all four would provide overdetermined fluxes weighted according to their noise and allow for consistency testing and gross error detection.

Analysis of the rate data by themselves (without reliance on a MFA model) show that rapamycin causes changes in central metabolism. Well-cultures given 100 nM of rapamycin, the previously determined optimum regarding resistance to cell death and enhanced specific productivity, had the largest glucose uptake rates and the lowest lactate-glucose ratios (FIG. 9). Using Model 2, such a shift in metabolism is estimated to correspond to relatively increased amounts of carbon flux through the TCA cycle (FIG. 11) and increased production of ATP (FIG. 10). Thus, we learned that the optimum concentration for beneficial physiological effects in our cell line is also the concentration at which we observed the most significant changes in metabolism, changes leading to increased uptake of and greater efficiency for glucose utilization in the TCA cycle. This provides us another aspect of rapamycin's effects on hybridoma physiology with which to continue investigating cell death and specific productivity in bioprocesses.

Prospective applications of HTMS span a wide range, including monitoring of metabolism of clones and inocula for bioprocess development, screening metabolism of cells in various media as part of medium design, and, more generally, merging metabolic information with genomic and proteomic data. The minimalist form of Model 2 should also aid in the development of novel micro-systems that would be otherwise unable to measure dozens of parameters simultaneously. Additionally, the simplified model and well-plate assays can be useful for teaching and learning metabolic flux analysis.

### Example 2

### 24-Well Plate pH and Acidification Rate Assay for Culture Mammalian Cells

In this example, a novel high throughput bioassay of evaluating in vitro cytotoxicity by real time monitoring acidification rate of fibroblast cells is developed. Rapid and precise real time pH measurement in a 24-well plate system is achieved by using pH indicator phenol red in combination with a spectrophotometric plate reader. Cell density is measured non-invasively with uv/vis spectroscopy by scanning multiple locations of each well. The method has been tested to quickly evaluate the in vitro cytotoxicity of 2,4-dinitrophenol and sodium fluoride. Results agree with the relative inhibition of medium acidification rate measured by the Molecular Devices Cytosensor. Medium acidification rate dependence on glucose and lactate metabolic rate is observed when cells are exposed to 2,4-dinitrophenol or fluoride. Comparing with other cytotoxicity evaluation methods, the microplate format and ease of detection reduces time consuming and costly steps in the process of drug detection. Among other things, it has the distinct advantage of allowing for multiple parallel measurements. Furthermore, the 24-well plate assay may be coupled to other measurements, enabling the evaluation of many parameters in a single experiment.

This rapid, high throughput pH assay can serve as a broad spectrum screen for changes in metabolism, and hence metabolic effects for any compound of interest. With regard to toxins, the assay can serve as a broad spectrum screen for cytotoxicity and be used as a parameter for toxin classification, discrimination, and/or identification.

### Materials and methods

Cell Culture and Media

Mouse fibroblast cells were obtained from ATCC(CRL-10225). During standard incubation, the cell line was maintained in DMEM (Mediatech) containing 10% fetal bovine serum (Sigma Chemical Co.) supplemented with final concentrations of 4 mM L-glutamine (Mediatech.), 10 U/ml penicillin-10 μg/ml streptomycin (Sigma Chemical Co.) in T75 flask at 37° C. under 10% CO₂.

Chemicals

Phenol red (the pH indicator), 2,4-dinitrophenol, and fluoride were purchased from Sigma Chemical Co. Trypan blue was purchased from Mediatech Co.

Estimation of Cell Density

Cell density of each well of a 24-well plate was accomplished by measuring the absorbance of a well at a wavelength of 560 nm and using a pre-determined calibration to convert absorbance to cell density. Absorbance readings for tests and calibration points were accomplished in a FL600 plate reader (Biotek Instruments) configured with appropriate absorbance filters. Using the multiscan capability of the KC4 software (Bioteck Instruments), the absorbance of each well was read in 25 different locations, and the average absorbance was used. This averaging was meant to reduce the effects of variations in cell coverage of the wells as well as edge effects.

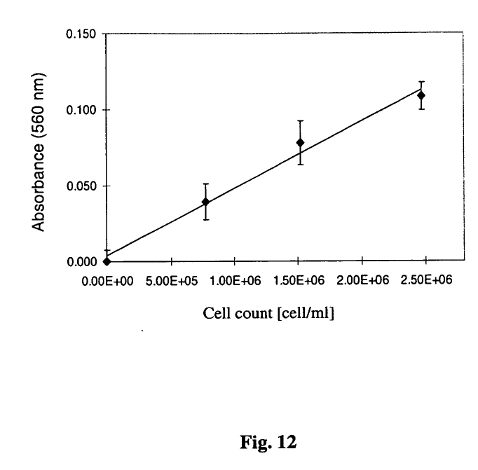

Absorbance measurements for the calibration curve were obtained from wells consisting of fibroblast cell cultures at cell densities of 5e5, 1.5e5 and 2.5e5 cells/ml. Cultures of each density were seeded into columns (4 wells) on a 24-well plate, as shown in FIG. 12, with one column reserved for medium as the blank. Prior to transferring cultures to the well plate, each culture was counted twice using trypan blue exclusion method (Mediatech) with hemocytometer. Cells were allowed to attach and medium was exchanged for test medium prior to scanning.

pH Monitoring and Estimation of Extracellular Acidification Rate

Without bounding to any theory, it is believed that pH indicator is normally used to visually estimate pH in mammalian cell culture, which also has been used to monitor hydrolase-catalyzed reaction accompanying pH change. Combined with a spectrophotometric plate reader, high throughput pH can be determined quantitatively. Combining Henderson-Hasselbach equation and phenol red dissociation equilibrium equation, relation between pH and absorbance can be expressed as equation (1).

$$pH = pKa + log \frac{O . D . - {A}_{min}}{{A}_{max} - O . D .} \qquad ( 1 )$$

To improve agreement of PH values with experimental measurements, the equation is modified using a parameter b to give

$$pH = pKa + b log \frac{O . D . - {A}_{min}}{{A}_{max} - O . D .} \qquad ( 2 )$$

Where Aₘᵢₙ and Aₘₐₓ are minimum and maximum absorbance of acid and basic form of phenol red indicator.

Design of Test Medium

Medium for 24-well plate pH assays (“test medium”) included low-buffered RPMI medium (Molecular devices), modified to contain 25 mg/ml phenol red and supplemented with 10 U/ml penicillin-10 μg/ml streptomycin (Sigma Chemical Co.) and 5 μg/ml insulin (Mediatech).

pH Assay

Fibroblast cells harvested from T75 flask at exponential phase were seeded into each well of 24-well plate (at 2e6 cells/ml) in 400 μl of standard medium and incubated at 37° C. One column contained only medium as the blank. After cells were attached to the well plate, the absorbance at 560 nm of each well on the plate was measured to estimate cell density. Then, standard medium was removed, wells were washed twice with PBS, and 600 μl of test medium was added to each well. In some cases, test medium for toxins having acid/base properties were equalized to 7.8 before adding into well plate. The column without cells was still used as blank, in the rest of five columns with cells, one toxicant free column was used as control, and other columns containing toxicants at four different concentrations were test columns. Place 24-well plate into the Fl 600 plate reader, whose temperature has been stabilized at 37° C. During 2 hours pH monitoring, the pH was measured every 18 minutes; multiple locations were still scanned in each well. There after, remove test medium and switch to toxicant free fresh medium to test the reversibility of toxicant effect, monitoring pH with additional 72 minutes. Finally, determine cell viability one well in each column using trypan blue exclusion method.

Glucose and Lactate Metabolic Rate

Glucose free RPMI medium (Sigma Chemical Co.) supplemented with 4 mM L-glutamine (Mediatech), 10 U/ml penicillin-10 μg/ml streptomycin (Sigma Chemical Co.) and 5 μg/ml insulin (Mediatech), was used as test medium. Attach cells (at 2e6 cells/ml) to each well of 24-well plate and determine cell density using same steps as medium acidification rate experiment. Switch maintenance medium to test medium containing toxicants at five different concentrations, toxicant free column used as control. Draw samples from each well to micro centrifuge tube, store in freezer for later analysis. Incubate well plate at 37° C. under 10% CO₂ for 6 hours, draw samples again for later analysis. Determine viability one well in each column using trypan blue exclusion method (Mediatech). Samples were used in performing the lactate (adapted from 826-UV Sigma assay protocol, Sigma) and glucose (adapted from 16-UV Sigma assay protocol, Sigma) assay.

### Results

Cell Density

Accurate determination of cell number is a common difficult problem presented in microplate experiments. Traditional methods, such as direct counting using hemocytometer, are too time-consuming and laborious to be used for high throughput applications. Many kinds of cell quantitation methods have been developed based on the activity of intracellular enzymes, such as esterase, cytosolic acid phosphatase, glyceroldehyde-3-phosphate dehydrogenase, and lactate dehydrogenase, where signals are obtained by incubating cells in defined periods in culture solutions containing an enzyme-specific substrate. Although high throughput determination of cell number can be obtained, each of these methods suffers from high variability over time. Furthermore, they can not be used in the studies of drugs which normally act on some enzymes. Using dyes binding to DNA overcome some of these limitations, but cumbersome sample preparation and damage of dye to cells limit their utilization. Noninvasive measurement of cell number can be obtained by measuring green fluorescence protein, it is unfortunate the utilization is limited to cells that constitutively express green fluorescence protein.

The direct noninvasive measurement of cell number of fibroblast using spectrophotometric is hindered by two factors, low absorbance and adherence. Cell attachment produces large absorbance variation at different locations of each well. Low absorbance significantly influences measurement precision. Scanning each well in multiple locations at high cell density helps mimize the effects of these limitations. As FIG. 12 shows, absorbance at 560 nm varied linearly with cell density over a range from 0 to 0.12 with R²=0.988. Error of cell density converted from absorbance is about 1e5 cells/ml. At each point, absorbance is the average of four wells, and absorbance of each well is the average of readings of 25 locations, where blank absorbance is subtracted from each well.

Real-Time pH Monitoring

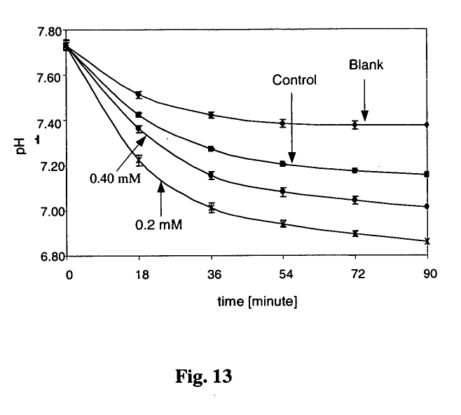

pH is a very sensitive parameter to temperature (data not shown) and environmental CO₂ (pH dropping of blank medium in FIG. 13). Precise measurement of pH can be obtained by incubating the 24-well plate at 37° C. in the plate reader. The influence of environmental CO₂ on low-buffered RPMI medium is small; pH sharp drift in the initial 18 minutes is caused mainly by temperature change. Absorbance produced from cells is subtracted before converting absorbance to pH values. Although low buffered RPMI medium is used, buffer capacity is still a factor blocking clear pH change. By adjusting pH of the medium to 7.8, which is away from buffer range, clear pH shift is observed when cells are exposed to toxicants at different concentrations as shown in FIG. 13.

Impact of Toxins on the Acidification Rate of Fibroblast Cells

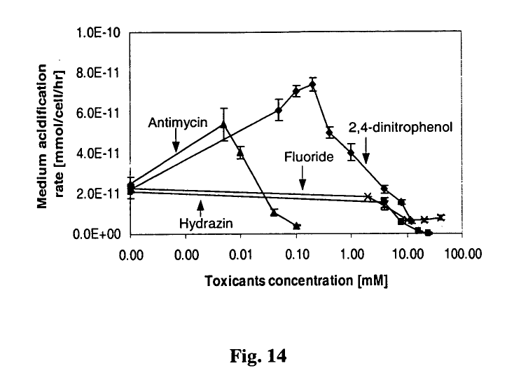

Medium acidification rate can be calculated using equation (3), where proton concentration changed in the blank has been subtracted. FIG. 14 shows the impacts of four toxicants at different concentrations on the medium acidification rate of fibroblast cells, each point represents mean±Stdev acidification rate of four wells.

$$q ( {H}^{+} ) = \frac{{C}_{{H}^{+}} ( t ) - {C}_{{H}^{+}} ( 0 )}{cell density * t} \qquad ( 3 )$$

The effects of all four kinds of toxicants on the acidification rate were concentration dependent. Among them, 2,4-dinitrophenol stimulate acidification rate at low concentrations in a concentration dependent manner until reaching a maximum value. Then with concentration increasing, stimulation effects become weak until inhibition effects are observed. Similar phenomenon was also observed for antimycin A. Both fluoride and hydrazine inhibit acidification rate in a concentration dependent manner. After 2 hours exposure to toxicants, acidification rate recovered to control level when switching to toxicant free fresh test medium except fluoride, where acidification rate only partially recovered (data not shown). Cells viability was all over 96% after toxicants exposure (data not shown). Medium acidification rate dependence on glucose, lactate metabolic rate

Glucose and lactate metabolic rates were determined from the total material balance around each well:

$$\frac{ⅆ C}{ⅆ t} = - {q}_{c} {n}_{V} \qquad ( 4 )$$

Yielding average metabolic rate:

$${q}_{c} = \frac{{C}_{f} - {C}_{0}}{{\overline{n}}_{v} \cdot ( {t}_{f} - {t}_{0} )} \qquad ( 5 )$$

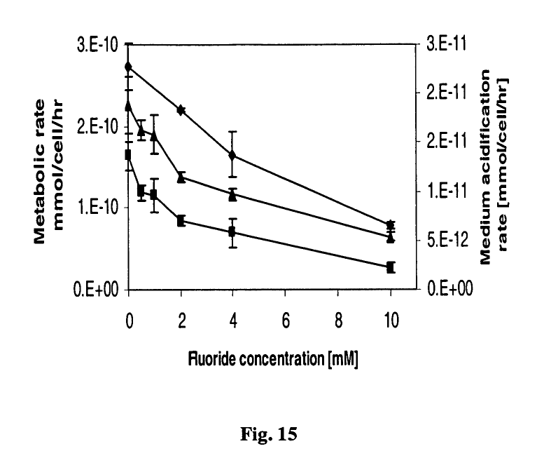

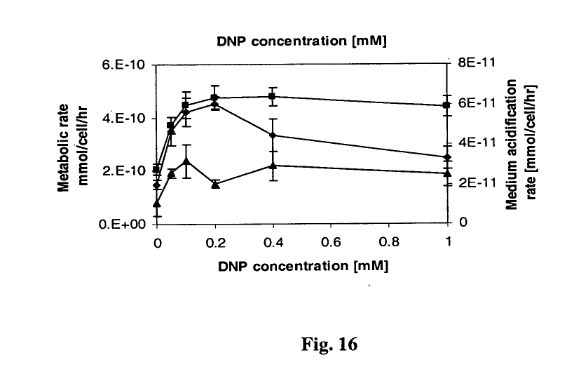

Both glucose and lactate metabolic rate were inhibited in concentration dependent manner when fibroblast cells were exposed to fluoride as shown in FIG. 15. For 2,4-dinitrophenol, an opposite impact was observed as shown in FIG. 16. Cell death was not observed when cells were exposed to 2,4-dinitrophenol. For fluoride, all cells died when fluoride concentration was at 10 mM. Medium acidification rate changes direction depending on glucose and lactate metabolic rate. Inhibition of glucose, lactate metabolic rate resulted in decreased acidification rate and vice versa, as shown in FIGS. 15 and 16.

While there has been shown various embodiments of the present invention, it is to be understood that certain changes can be made in the form and arrangement of the elements of the system and steps of the methods to practice the present invention as would be known to one skilled in the art without departing from the underlying scope of the invention as is particularly set forth in the Claims. Furthermore, the embodiments described above are only intended to illustrate the principles of the present invention and are not intended to limit the claims to the disclosed elements.

What is claimed is:

1. A method for determining at least one metabolic rate of a plurality of cells, comprising the steps of:
a. providing a first plate having a plurality of wells, each well having a bottom and side portions defining a volume and an opening opposite the bottom, wherein the total number of the plurality of wells is L, L being an integer;
b. placing a solution of medium and cells in one or more wells of the first plate, wherein the amount of solution in each well in terms of volume is v₀;
c. withdrawing a first volume, v₁, of medium with or without cells from one or more wells of the first plate, thereby leaving a second volume, v₂, of medium and cells in one or more wells of the first plate;
d. incubating the first plate for a period of time, T₁;
e. withdrawing a third volume, v₃, of medium with or without cells, from one or more wells of the first plate, thereby leaving a fourth volume, v₄, of medium and cells in one or more wells of the first plate;
f. withdrawing a fifth volume, v₅, of medium with cells, from one or more wells of the first plate, thereby leaving a sixth volume, v₆, of medium and cells in one or more wells of the first plate;
g. obtaining cell-free solutions from the first and third volumes;
h. using the cell-free solutions in an assay;
i. measuring the concentration of at least one metabolite in the first and third volumes or in the second volume at least two times within a time period T₂, wherein T₂ is less than or equal to T₁ and within time period T₁; and
j. determining at least one metabolic rate for the metabolite measured for each of one or more wells of the first plate that contained a plurality of cells from the measured concentration of at least one metabolite.

2. The method of claim 1, wherein the first plate comprises a well-plate and L is 24.

3. The method of claim 1, wherein original volume v₀ is smaller than 1,000 μl.

4. The method of claim 1, wherein the cells grow in suspension remaining unattached from the bottom or side surfaces.

5. The method of claim 4, wherein the step of obtaining cell-free solutions comprises the step of centrifugating the first volume and the third volume, respectively.

6. The method of claim 1, wherein the cells grow attached to the bottom or side portions of the well or on a device placed in the well.

7. The method of claim 6, wherein the step of obtaining cell-free solutions comprises the step of avoiding the cells attached to the bottom or side portions of the well or a device placed in the well.

8. The method of claim 7, wherein the device placed in the well comprises a scaffold or at least one microcarrier.

9. The method of claim 1, prior to the step of withdrawing a first volume, further comprising the step of keeping the solution and the cells in one of more wells of the first plate for a period of time, T₃.

10. The method of claim 9, wherein T₃ is sufficiently long to allow adherent cells to attach to a surface of a corresponding well or a device placed therein.

11. The method of claim 1, wherein the incubating step further comprises the step of placing the first plate in an incubator.

12. The method of claim 1, prior to the step of placing a solution of medium and cells in one of more wells of the first plate, further comprising the step of preparing the solution of medium and cells in a parent culture.

13. The method of claim 1, subsequent to the step of obtaining cell-free solutions, further comprising the step of storing the cell-free solutions for later use.

14. The method of claim 13, wherein the cell-free solutions is stored in a refrigerator.

15. The method of claim 13, wherein the cell-free solutions is stored in a freezer.

16. The method of claim 1, subsequent to the step of withdrawing the fifth volume, further comprising the step of performing a cell count to determine cell concentration and culture viability from a portion of the fifth volume.

17. The method of claim 1, subsequent to the step of withdrawing the fifth volume, further comprising the step of performing an assay for apoptosis and necrosis.

18. The method of claim 1, subsequent to the step of withdrawing the fifth volume, further comprising the step of performing a cellular or molecular biology assay.

19. The method of claim 1, wherein a plurality of metabolic rates of the cells are determined, the total number of the plurality of metabolic rates being an integer Q.

20. The method of claim 19, wherein at least one of the plurality of metabolic rates is for consumption or production of glucose, lactate, any of amino acids, oxygen, carbon dioxide, hydrogen ion (pH), or biopharmaceutical.

21. The method of claim 1, wherein the solution of medium and cells in each well of the first plate has a cell density substantially similar to each other.

22. The method of claim 21, wherein the cell density of the solution of medium and cells in each well of the first plate is in the range of 1.0×10⁴ to 1.0×10⁹ cells/ml.

23. The method of claim 22, wherein the cell density of the solution of medium and cells has a concentration of cells of about 2.0×10⁶ cells/ml.

24. The method of claim 21, wherein the amount of biological entity in the solution is in the range of 0.0001 to 2000 grams/liter.

25. The method of claim 1, wherein the solution of medium and cells in each well of the first plate has a cell concentration different from each other.

26. The method of claim 1, further comprising the step of supplying a number of cells to each well of the first plate.

27. The method of claim 1, further comprising the step of supplying an amount of medium to each well of the first plate.

28. The method of claim 1, further comprising the step of analyzing the first and third volumes obtained from each well for at least one metabolite concentration.

29. The method of claim 28, wherein the step of analyzing comprises the following steps:
a. providing at least one second plate having a plurality of wells, each well having a bottom and side portions in cooperation defining a volume and an opening opposite the bottom, wherein the total number of the plurality of wells is M, M being an integer larger than L; and
b. placing portions the solution from one or more volumes obtained from the first plate into each of S wells of at least one second plate, wherein each of S wells of at least one second plate contains a reagent solution for accomplishing a particular metabolite assay for R times, where R is an integer and S is an integer smaller than M.

30. The method of claim 29, wherein the volumes of the solution used are the first volumes from the first plate, and the number of wells needed in at least one second plate is no greater than R×L where each volume is apportioned R times.

31. The method of claim 29, wherein the volumes of the solution are the third volumes from the first plate, and the number of wells needed in at least one second plate is no greater than R×L where each volume is apportioned R times.

32. The method of claim 29, wherein the volumes of the solution are both the first and third volumes from the first plate, and number of wells needed in at least one second plate is no greater than 2×R×L where each volume is apportioned R times.

33. The method of claim 29, wherein the metabolite analyzed is glucose and the reagent solution contains enzymes and substrates that use glucose to create NADPH.

34. The method of claim 29, wherein the metabolite analyzed is lactate and the reagent solution contains enzymes and substrates that use lactate to create NADH.

35. The method of claim 29, wherein the metabolite analyzed is carbon dioxide and bicarbonate and the reagent solution contains enzymes and substrates that use bicarbonate to oxidize NADH.

36. The method of claim 29, wherein M is at least three times larger than L.

37. The method of claim 29, wherein L is 24 and M is 96.

38. The method of claim 29, wherein R is 3.

39. The method of claim 1, prior to the step of withdrawing a first volume, further comprising the step of monitoring the pH of each well in the first plate by spectroscopy for a time period T₄, which is less than or equal to T₂.

40. The method of claim 39, wherein one or more wells of the first plate are sealed during T₄.

41. The method of claim 39, prior to the step of withdrawing a first volume, further comprising the step of monitoring the oxygen concentration of each well by spectroscopy for a time period T₅, which is less than or equal to T₂, and may overlap with or coincide with T₄.

42. The method of claim 41, wherein one or more wells of the first plate are sealed during T₅.

43. The method of claim 41 further comprising the step of sampling a seventh volume, v₇, and an eighth volume, v₈, from one or more wells of the first plate immediately before and immediately after a period of time, T₆, which is less than or equal to T₂ in length, and may overlap with or coincide with at least one of T₄ and T₅, to leave volumes v₉ and v₁₀ in one or more wells of the first plate, respectively.

44. The method of claim 43, wherein one or more wells of the first plate are sealed during a period of time T₆.

45. The method of claim 1, wherein the determining step further comprises the step of determining at least one or more amino acids from portions of the first and third cell-free volumes.

46. The method of claim 45, wherein the step of determining at least one or more amino acids further comprises the step of determining amino acids by using a liquid chromatography system.

47. The method of claim 1, wherein the determining step further comprises the step of determining biopharmaceutical concentration from portions of the first and third cell-free volumes.

48. The method of claim 47, wherein the biopharmaceutical comprises a monoclonal antibody.

49. The method of claim 47, wherein the biopharmaceutical comprises a therapeutic protein.

50. A method for calculating at least one unknown metabolic flux of a plurality of cells, comprising the steps of:
a. constructing a metabolic network having a plurality of reaction components, the reaction components representing at least glycolysis, reduction of pyruvate to lactate, TCA cycle, and oxidative phosphorylation;
b. measuring at least two metabolic rates of a plurality of cells corresponding to at least two of the metabolic network reactions; and
c. calculating metabolic fluxes of a plurality of cells for the rest of the metabolic network reactions from at least two measured metabolic rates of a plurality of cells corresponding to at least two of the reactions.

51. The method of claim 50, further comprising the steps of:
a. measuring at least one additional metabolic rates of a plurality of cells corresponding to an additional one of the reactions;
b. constructing a set of equations that are overdetermined for the metabolic rates of a plurality of cells for the reaction components; and
c. calculating metabolic fluxes of a plurality of cells for all of the reactions from the set of equations.

52. The method of claim 50, further comprising the step of feedbacking the measured at least two metabolic rates of a plurality of cells corresponding to two of the reaction components from the determined metabolic rates.

53. The method of claim 50, wherein the plurality of reaction network components include glucose, pyruvate, lactate, CO₂, O₂, ATP, NADH, FADH₂, and amino acids.

54. The method of claim 50, wherein measurable reaction fluxes include glucose, lactate, oxygen, and carbon dioxide metabolic rates, and calculated fluxes include glycolysis, TCA cycle, oxidative phosphorylation, and ATP production.

55. A system for calculating at least one unknown metabolic flux of a plurality of cells, comprising:
a. means for constructing a metabolic network having a plurality of reaction components, the reaction components representing at least glycolysis, reduction of pyruvate to lactate, TCA cycle, and oxidative phosphorylation;
b. means for measuring at least two metabolic rates of a plurality of cells corresponding to at least two of the metabolic network reactions; and
c. means for calculating metabolic fluxes of a plurality of cells for the rest of the metabolic network reactions from at least two measured metabolic rates of a plurality of cells corresponding to at least two of the reactions.

56. The system of claim 55, further comprising:
a. means for measuring at least one additional metabolic rates of a plurality of cells corresponding to an additional one of the reactions;
b. means for constructing a set of equations that are overdetermined for the metabolic rates of a plurality of cells for the reaction components; and
c. means for calculating metabolic fluxes of a plurality of cells for all of the reactions from the set of equations.

57. The system of claim 55, further comprising means for feedbacking the measured at least two metabolic rates of a plurality of cells corresponding to two of the reaction components from the determined metabolic rates.

58. The system of claim 55, wherein the plurality of reaction network components include glucose, pyruvate, lactate, CO₂, O₂, ATP, NADH, FADH₂, and amino acids.

59. The system of claim 55, wherein measurable reaction fluxes include glucose, lactate, oxygen, and carbon dioxide metabolic rates, and calculated fluxes include glycolysis, TCA cycle, oxidative phosphorylation, and ATP production.

60. The system of claim 55, wherein the measuring means comprises a first well plate having a plurality of wells, each well having a bottom and side portions in cooperation defining a volume and an opening opposite the bottom, wherein the total number of the plurality of wells is L, L being an integer.

61. The system of claim 60, wherein the measuring means further comprises a second well plate having a plurality of wells, each well having a bottom and side portions in cooperation defining a volume and an opening opposite the bottom, wherein the total number of the plurality of wells is M, M being an integer.

62. The system of claim 61, wherein L is different from M.

63. The system of claim 61, wherein L equals M.

64. The system of claim 55, wherein the calculating means comprises a controller.
